(19) United States
(12) Patent Application Publication
(10) Pub. No.: US 2025/0176768 A1
(43) Pub. Date: Jun. 5, 2025

(54) CLEANING SYSTEMS AND METHODS

(71) Applicant: Good Mother Earth Cleaning, Inc., Las Vegas, NV (US)

(72) Inventors: Jon Orban, Las Vegas, NV (US); Alvaro Siman, Imperial Beach, CA (US)

(21) Appl. No.: 19/037,179

(22) Filed: Jan. 25, 2025

Related U.S. Application Data
(63) Continuation of application No. PCT/US2023/028181, filed on Jul. 19, 2023.
(60) Provisional application No. 63/576,529, filed on Feb. 15, 2023.
(60) Provisional application No. 63/576,327, filed on Feb. 1, 2023.
(60) Provisional application No. 63/475,990, filed on Jan. 5, 2023.
(60) Provisional application No. 63/474,532, filed on Aug. 22, 2022.
(60) Provisional application No. 63/474,170, filed on Jul. 26, 2022.

(51) Int. Cl. A47K 7/04; A61L 2/00; A61L 2/025; A61L 2/10; A61L 2/18; A61L 2/20; A61L 2/24; A61L 101/02; A61L 101/34

(52) U.S. Cl. CPC A47K 7/04; A61L 2/0047; A61L 2/0088; A61L 2/0094; A61L 2/025; A61L 2/10; A61L 2/18; A61L 2/20; A61L 2/202; A61L 2/24; A61L 2101/02; A61L 2101/34; A61L 2202/11; A61L 2202/121; A61L 2202/122; A61L 2202/14; A61L 2202/15; A61L 2202/17

(57) ABSTRACT

Cleaning systems and methods, wherein the system includes a chamber configured to receive a body part of a user or an object, where the user's skin or object is exposed to one or more cleaning, disinfecting and/or sterilizing processes, such as chemical agents, mechanical energy, ultraviolet light, and ultrasound, to clean, disinfect and/or sterilize the skin or object.

### CROSS-REFERENCE TO RELATED APPLICATIONS

This application is a continuation under claims 35 U.S.C. § 120 to International Application no. PCT/US2023/028181 filed Jul. 19, 2023, entitled “Cleaning Systems and Methods,” which claims priority to and benefit under 35 U.S.C. § 119(a)-(e) to U.S. Provisional Application filed Jun. 20, 2023, entitled “Network of Reprogrammable Self-Sanitizing Architectural Components,” U.S. Application No. 63/576,529 filed Feb. 15, 2023, entitled “Novel Design Strategies for a Self-Disinfecting Doorknob and Other Architectural Components,” U.S. Application No. 63/576,327 filed Feb. 1, 2023, entitled “Alternate Embodiment and Improvement of Novel Hand Sanitizing Machine,” U.S. Application No. 63/475,990 filed Jan. 5, 2023, entitled “Novel Strategies and Combination of Processes for Automatic Scrubbing and Disinfecting Machines,” U.S. Application No. 63/474,532 filed Aug. 22, 2022, entitled “Novel Design of a Multi-Process And Automatic Disinfecting Machine,” and U.S. Application No. 63/474,170 filed Jul. 26, 2022, entitled “Novel Design of an Automatic Scrubbing And Disinfecting Machine,” all of which are hereby incorporated by reference in their entirety as part of the present disclosure.

### FIELD OF THE INVENTION

The invention relates cleaning systems and methods, including systems and methods for cleaning or sanitizing a user's skin and external tissues and optionally jewelry, accessories and objects that may contact the user's skin. The systems and methods may be part of an early detection and warning and/or tracking network for pathogen outbreaks or pandemics, and outbreak/pandemic response/monitoring systems.

### BACKGROUND

The benefit and need to clean or sterilize a person's skin is well-understood and ubiquitous in modern human society. Doing so helps prevent contaminants and deleterious microbials from injuring or sickening the person, and helps prevent transferring such contaminants/microbials to other persons and objects. In addition to health concerns, the introduction of contaminants into industrial or commercial settings can be detrimental, if not disastrous, to the quality and operation of the products and/or services provided.

Nonetheless, humans still clean/sterilize their skin much the same way they have for hundreds of years. More specifically, the processes involved remain largely manual in nature and vaguely-defined. The effectiveness of such processes is thus highly subject to an individual's knowledge, consistency and diligence, and in some instances, the physical and/or mental capabilities of the individual person. In this regard, different societal populations and sectors have different or unique standards of cleanliness. It is also impossible to eliminate human error, and to a significant degree, random chance that can affect whether a sufficient cleanliness level is achieved.

As but one example, the importance of pre-surgical scrubbing by surgical theater personnel to help prevent surgical patient infection is well-known and widely-practiced. However, there are many factors that go into the effectiveness of such, including, but not limited to, the cleaning products and tools used, the scrubbing time, and the specific technique used, in addition to the “human” factors discussed above. Ultimately, even something as important as pre-surgical scrubbing is subject to significant discretion of individual person, creating a non-standard and uncontrolled variation of outcomes to this mission-critical aspect of surgical theater procedures.

Yet more, there exists a risk of reinfection from the various accessories that a person uses or encounters subsequent to disinfection. Common examples include, but are not limited to, jewelry, electronic devices (mobile phones) and hand-held tools.

Conversely, though the COVID pandemic brought significant focus on cleaning and disinfection, it also brought on overuse and misuse of various sterilizing methods, including the use of common sterilizing agents. Sterilizing fluids containing methanol, alcohol (ethanol), benzene, acetaldehyde, acetol, methanol, 1-propanol, and benzene are toxic. Their increased use a consequence of COVID—both in frequency and quantity—raised significant health concerns, so much so that the U.S. Food and Drug Administration released a list of products to avoid using and even recalled some products. Moreover, sanitizer use can result in antimicrobial drug resistance. Furthermore, many sanitizer chemicals are caustic, and can damage products, including various electronic devices, eyewear, tools, and access control devices, and products that have specially engineered coatings or casings that can be permanently damaged if sterilized with the above-mentioned disinfectants. Accordingly, use of sterilizing agents as was done during the pandemic can no longer be regarded a viable option for keeping the human population safe from microbial vectors, even as a stopgap measure when a pandemic threatens.

### SUMMARY

Cleaning, sanitizing and sterilizing devices and methods use a combination of processes to achieve significantly enhanced disinfection, including combinations of chemical, ultraviolet, ultrasonic, and, in at least some embodiments, mechanical processes. Their strategic combination substantially speeds up disinfection to an unexpected speed and efficiency. At the same time, safety to humans is increased. As such, the invention provides a more sustainable method of protecting the human population in the various societal interactions in which they may participate.

Devices utilizing the invention can automate and standardize the disinfection process. Body parts inserted into such devices undergo a programmed cycle of chemical, ultraviolet, ultrasonic, and, in at least some embodiments, mechanical processes to disinfect the skin of such body parts. The automation and/or sterilization can reduce human error, different outcomes based on differences among users, and the effect of external events on the effectiveness of the treatment.

The sanitization course can be targeted to a contaminant or microbial of concern, or to the health of the user or the needs of the commerce or industry in which user participates. The devices may be connected to or connectable to a network or networks that are local and/or global in nature, so as to receive updates based on current conditions or projections, and the disinfection cycle adjusted as needed to address the same.

In another aspect, the networked devices can function as part of an early detection and warning infrastructure for pathogen outbreaks or pandemics. Biosensors in the devices, e.g., devices installed in schools, courtrooms, and other high-traffic public spaces, can detect pathogens and communicate information regarding same to local authorities such as first responders, local hospitals, and local government, as well as regional, national and/or global authorities. In further aspects, such networks can assist authorities in monitoring pathogen spread and allocation of resources.

Other objects and advantages of the present invention will become apparent in view of the following detailed description of the embodiments and the accompanying drawings.

### BRIEF DESCRIPTION OF THE DRAWINGS

Embodiments will be described below with reference to the drawings. However, those skilled in the art should appreciate that the drawings are only for the purpose of explaining the described embodiments, and therefore do not limit the scope of the invention or the claims herein. In addition, unless otherwise indicated, the drawings are intended only to conceptually represent the described embodiments, and are not necessarily drawn to scale.

FIG. 1A is a schematic front view of an embodiment of a cleaning apparatus;

FIG. 1B is schematic side view of the embodiment of FIG. 1A;

FIGS. 2A and 2B are schematic representations of the complimentary effect of using multiple disinfection vectors as described herein;

FIG. 3 is a schematic representation of a network of multi-process devices and architectural elements;

FIG. 4A is a schematic front view of another embodiment of a cleaning apparatus; FIG. 4B is schematic side view of the embodiment of FIG. 4A;

FIG. 4C is a schematic top view of a user using the embodiment of FIG. 4A;

FIG. 5A is a schematic top view of another embodiment of a cleaning apparatus;

FIG. 5B is schematic front view of the embodiment of FIG. 5A;

FIG. 5C is schematic front perspective view of the embodiment of FIG. 5A;

FIG. 5D is another schematic top view of the embodiment of FIG. 5A;

FIGS. 6A-6C show a schematic side view of another embodiment of a cleaning apparatus;

FIG. 7 is a schematic front view of another embodiment of a cleaning apparatus;

FIG. 8 is a schematic front view of another embodiment of a cleaning apparatus;

FIG. 9 is a schematic left view of another embodiment of a cleaning apparatus;

FIG. 10 is a schematic left view of another embodiment of a cleaning apparatus;

FIG. 11A is a schematic front view of another embodiment of a cleaning apparatus;

FIG. 11B is a schematic side view of the embodiment of FIG. 11A;

FIG. 11C is a schematic front perspective view of the embodiment of FIG. 11A;

FIG. 11D is a schematic top view of the embodiment of FIG. 11A during use;

FIG. 12A is a schematic depiction of an embodiment of mechanical scrubber;

FIG. 12B is a schematic depiction of the mechanical scrubber of FIG. 12A in use;

FIG. 13A is a schematic depiction of a further embodiment of mechanical scrubber; and

FIG. 13B is schematic depiction of the mechanical scrubber of FIG. 13A in use.

### DETAILED DESCRIPTION

Embodiments are described below with reference to the drawings. It should be noted that like numerals in separate drawings represent like items. Therefore, once a certain item in a drawing is described, it might not be further defined and explained in the subsequent drawings. In addition, it should be noted that orientations, positions and relationships discussed herein, e.g., terms such as “front”, “rear” and the like, are provided solely to facilitate the description of the respective embodiment, and do not indicate that a particular orientation is required, or that the invention must be configured and/or operated in the particular orientation, and thus should not be construed as limiting the scope of the invention and claims herein.

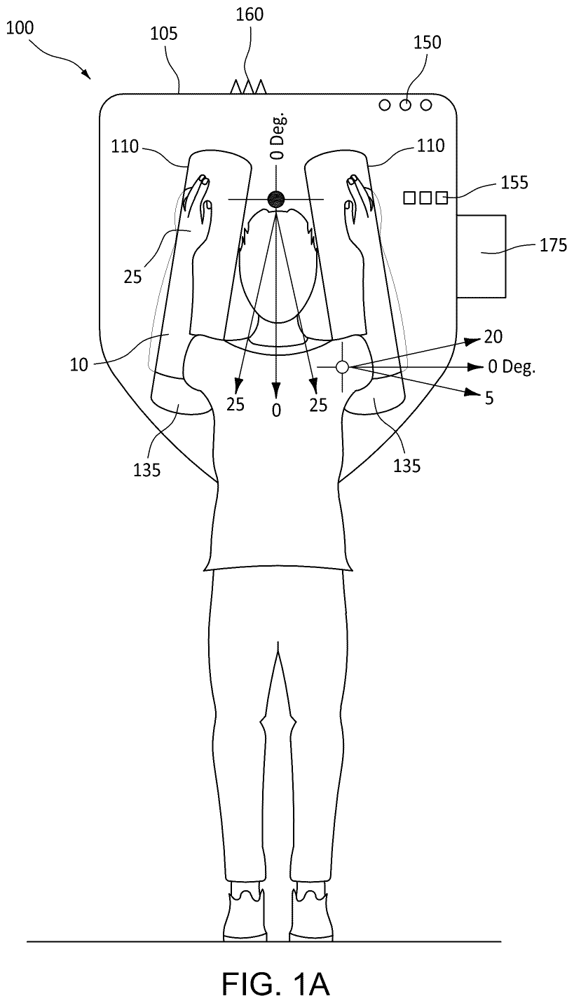

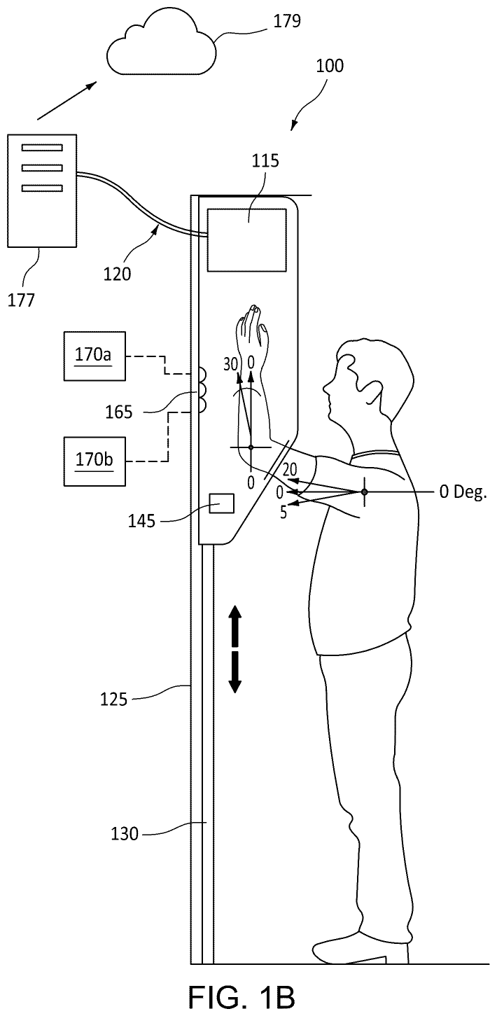

FIGS. 1A and 1B show an embodiment of cleaning, disinfection and/or sterilization apparatus 100. The unit 100 includes an enclosure 105 that hold or generally encloses the unit's components and includes one or more chambers or tunnels 110 adapted to receive body parts of a user, such as appendages. The enclosure 105 can be formed of any suitable material. However, embodiments made from durable and/or more easily cleanable materials, e.g., plastic, steel or stainless steel panels, are advantageous. Moreover, the enclosure 105 can be made of or contain, e.g., a coating, antimicrobial materials, such as, for example only, antimicrobial polymers, copper, and/or silver. The enclosure 105 has an interface 115 for communication with a user and/or unit operators. The interface 115 can be, for example, a control panel, a screen, a touchscreen, a keypad/keyboard, audio input(s)/output(s) (e.g., microphone(s) and speaker(s)), or any combinations of the foregoing. A signal connection 120 operatively connects the interface 115 to a least one external server 177 or other component for signal communication therebetween. The at least one external component may be a computer, computer servers, a network, the Internet, the Cloud 179, etc. Further, though the signal connection 120 shown in FIG. 1B is depicted as a wired connection, it may be any suitable connection, wired or wireless, e.g., WiFi, Bluetooth, cellular, RF, etc.

The enclosure 105 is connected to and supported by a support 125 that is connected to the floor. Though the illustrated embodiment contains a floor-stand type support, in other embodiments the support can be mounted to a wall, or supported by some other structure. The support 125 defines a rail 130 that is configured to permit the height of the enclosure 105 to be adjusted by movement thereof along the rail 130. The movement may be achieved by any suitable mechanism, such as those that are currently known or become known, and the movement may made manually (by a person) or be powered, e.g., by motors, pneumatically, hydraulically, etc. The unit 100 may include a mechanism, such as a lock mechanism or detent, to maintain the enclosure 105 at a selected height during use. In at least some embodiments, the height may be adjusted by a user or operator via the interface 115. For example, the height can be manually input or obtained by the interface 115 from user information stored an access card or a SaaS or GME database on the local or wide area network (not shown) operatively connected to the interface 115 via the signal connection 120.

The enclosure 105 further defines openings 135 provide access to respective chambers 110. The chambers 110 and openings 135 thereto are each constructed with both a size and configuration/shape to receive therein a broad range of body parts the chambers 110 are intended to receive. In the illustrated embodiment, the chambers are each configured to receive a significant portion of the arm 10 of a user 5, and thus are configured to receive a broad range of diameters, lengths and diameters of users' elbows, forearms, wrists and hands 25. In at least some embodiments, the tunnels 110 may be adjustable to a particular user's body part (e.g., hand, forearm, upper arm, and elbow length and girth), such as by a mechanical scissor, fan, or rotary expandable and collapsible tunnel structure system. The tunnel aperture could be comprised of sections of tubes wrapped in a flexible and stretching outer membrane where each section is connected to a scissor jack mechanism that dilates the aperture via motorized controllers. Alternatively, the tunnel aperture may be comprised of a series of tubular sections with slots that mechanically dilate the tunnel aperture as a motor turns the gear and slot mechanism. This tunnel aperture could also be made to dilate by having the inside tubular sections be inside of a conical outer tube, joined by slotted tracks. The sliding of the inner and outer tube would close and open the inner tube by sliding it up and down the conical inner geometry of the outer tube. Such adjustment may be made manually, e.g., by a user or operator, or, similarly to the height adjustment discussed above, via the interface 115 by user input thereto or using user information stored in an access card or a SaaS or GME database on a local or wide area network.

As seen, the illustrated embodiment has two tunnels 110 for receiving two body parts, i.e., a user's arms 10. Other embodiments have a single tunnel 110, for receiving a single body part. Such embodiments may be used, for example, where only a single body part requires cleaning/disinfection, where a smaller enclosure is desired or necessary, or where a less expensive device is desired, in which case body parts can be disinfected sequentially. Single chamber 110 units may be used, for example, for a less abled user, e.g., a user that has only one hand or arm. Nonetheless, dual chamber embodiments may also be utilized for such users, and in some such embodiments, one of the tunnels 110 can be “turned off” during use.

In the illustrated embodiment, the enclosure 105 further includes seals 140 that engage the user's body part when received within the tunnel 110. The seals 140 may comprise inflatable cuffs or bladders that can be transformed between a deflated condition that permits a user to insert the body part through the aperture 135 and into the tunnel 110, and an inflated condition where it engages the body part. Such seals may be inflated, for example, by filling the cuff or bladder with air, similar to a blood pressure testing cuff, or with a pressurized liquid. The air or liquid may be moved to and from the cuff or bladder by a pump 145 or similar device. In FIG. 1B, the pump 145 is located inside the enclosure 105 but may alternatively be located outside the enclosure 105.

In other embodiments, the seal 140 may comprise a flexible and/or compressible material that, after the user inserts the body part through the opening, the seal engages the body part. In some such embodiment, the seal 140 may comprise a neoprene or rubber ring and including anti-microbial polymer(s).

In at least some embodiments, the seals 140 are configured to engage the body part with sufficient contact and force against the skin to so as to at least substantially seal the chamber 110 from the surrounding environment outside the chamber 110. Also in at least some embodiments, the seals 140 can include antimicrobial material(s) or coating(s), e.g., antimicrobial polymer(s).

In yet other embodiments. the seals 140 may be formed by walls of pressurized air that isolates the interior of the chamber 110 from the exterior by pneumatic force.

The cleaning/disinfection/sterilization of the user's skin takes place in the chambers 110 via combination of processes, including ultraviolet light, ultrasound, anti-microbial agents, and, in at least some embodiments, mechanical methods.

The user's skin can be exposed to sterilizing UV-A, UV-B, and/or UV-C light. UV-C radiation has been shown, for example, to destroy the outer protein coating of the SARS-Coronavirus, including wavelengths of 254 nm and 222 nm. UV-C is presently considered the safest for human use, with the 222 nm wavelength being considered safer than the 254 nm wavelength. The chamber 110 includes UV emitters 150 (e.g., UV “lights”) that emit UV light at one or more selected frequencies and irradiate the skin for selected exposure time(s), thus irradiating microbials located on or near the skin surface (as well as, for example, on skin hairs).

The chamber 110 also includes ultrasound emitters 155 (e.g., speakers) that emit ultrasound at one more selected frequencies and expose the skin to the sound. Ultrasonic and harmonic resonance damages or kills viruses and contagions by bursting or crushing the lipid bi-layer virus shell wall, and/or, in the case of a “corona” type virus, crush or dismember its “corona” spikes. Crushing or dismembering the “corona” spikes is effective, as it is through these spikes that viruses bind with their cellular counterparts of the invaded/infected human cell as well as the initiation of the transfer of its RNA genome into the victim cell. Various known contagion culprits, including, but not limited to the HIV, influenza, and the novel SARS-CoV-2 strains have a similar shell/spike structure and are thus vulnerable to harmonic excitation attacks.

Frequencies between about 20-100 megahertz are effective in debilitating or destroying such contagions. These ultrasound frequencies are considered to be safe to the human body. Of note is the speed by which these resonant frequencies damage or weaken the contagion(s). Vibrations between about 25 and 100 megahertz begin to rupture the virus shell or “corona” spike within a fraction of a millisecond. This desired effect is reached at only 0.3 microseconds at a broad range of frequencies. This means that this can be done by having the machinery cycle through a multiplicity of target frequencies in a single user session. Thus, it is possible to program a series of strategically chosen pulses to sweep over a broad range of frequencies (in conjunction and combination with UV and other chemical and mechanical processes) to accelerate sterilizing efficiency at arithmetic rates. In at least one embodiment, the sterilizing unit 100 cycles between either randomized or selected combinations of 110 MHz, 50 MHZ, and 25 MHz frequencies. As should be appreciated by those of ordinary skill in the art, though, the frequencies and cycles can be tailored to target specific sterilization needs, e.g., of the user, for current health concerns (e.g., a particular current spreading or widespread contagion), for differing societal sectors or commercial industries, etc.)

Further, Ultrasound and UV in combination with high-speed turbulent ionized air have been found effective in vibrating and tumbling away dirt and contagions (as they are exposed to damaging/deadly light and sound energy). Accordingly, the unit 100 contains Ultrasound emitters 160 to vibrate the enclosure 105 at target frequencies to sanitize the external surface of the enclosure 105. Such mitigates the potential for post-sterilization re-contamination of the user's skin by contact with the outside of the enclosure 105.

The chamber 110 also includes opening or nozzles 165 that dispense on or more anti-microbial agents into the chamber 100 and onto the user's skin, thus damaging or killing (alone or in combination with Ultrasound and/or UV) microbials thereon. The agents are delivered from agent sources 170a, 170b in or placeable in fluidic connection (see the broken lines in FIG. 1B) with the nozzles 165. Though FIG. 1B shows two sources, other embodiments can have fewer or more sources. In addition, though the sources 170a, 170b are shown located outside the enclosure 105, in other embodiments they are located inside the enclosure.

The microbial agents can comprise any suitable agent, either currently known or later becomes known. Antiseptic ST37, also known as hexylresorcinol, reduces the surface tension of a virus' cell wall, weakening it. Its combination with other anti-microbial processes, e.g., UV, Ultrasound and/or mechanical means increases the overall kill effectiveness exponentially. For example, exposing the ST37 weakened cell wall with UV-C and resonant sound frequency vastly increases breaking and/or bursting of the cell wall, killing the virus.

Ozone gas may also be used. As previously described, bacteria are single-cell, simple structure, microorganisms with an outer membrane. This membrane is particularly susceptible to exposure to ozone. The membrane is broken down by exposure to ozone relatively quickly, thus significantly debilitating the bacteria, making it susceptible final destruction when exposed to one or one of the other processes discussed herein. Ozone also destroys most viruses by diffusing through the protein coating of the outer membrane into the nucleic acid core. This damages the RNA of the virus (ribonucleic acid). This process happens rapidly with some pathogens destroyed in seconds with adequate ozone levels, which can set to target the pathogens of concern, e.g., the a particular industry or application. Ozone also provides an advantage over other disinfectants as it is able to disinfect objects within a volume of air and not just surfaces, rapidly and with no danger to humans at the levels used.

Nitric oxide gas (NO) can also be used. NO is a free radical and short-lived, diatomic, lipophilic gas that can be leveraged in attacking pathogens. Depending on its concentration, NO exerts antimicrobial effects in two ways. At low concentrations, NO acts as a signaling molecule that promotes the growth and activity of immune cells. NO signaling directs a broad spectrum of processes, including the differentiation, proliferation, and apoptosis of immune cells. NO can also directly destroy pathogens that have been taken up by phagocyte, a type of cell that has the ability to ingest, and sometimes digest, foreign particles, such as bacteria. Moreover, at higher concentrations, NO covalently binds DNA, proteins and lipids, thereby inhibiting or killing target pathogens. This is due to the fact that NO is a lipophilic and hydrophilic natural gas, with a small Stokes radius that allows it to cross membranes readily. Yet further, as a radical gas, (gNO) is unstable in an oxygen environment. Reactions of gNO with oxygen or superoxide spontaneously produce reactive nitrogen and oxygen intermediates that lead to the formation of a variety of antimicrobial species. The formation of these intermediates becomes biologically significant when the concentration of NO is greater than 1 μM. At these concentrations, reactive nitrogen oxide species (RNOS) causes oxidative and nitrosative damage by altering DNA, inhibiting enzyme function, and inducing lipid peroxidation, which account for the majority of NO's antimicrobial properties. A specialized chamber, like chamber 110, can deliver gNO safely and securely, especially when the seal 140 seals the chamber 110. The chamber 100 allows for controlled delivery of NO and prevents oxygen species from reacting with the NO to produce toxic NO₂. At a concentration of 160 ppm, gNO becomes actively bactericidal.

The above are only examples of microbial agents that may be used. Those of ordinary skill in the art will appreciate other agents that may be utilized with the invention.

As discussed above, the nature of ever-evolving viruses and bacteria means that strains may become resilient to a particular type of sanitization, e.g., UV, Ultrasound or chemical agents. However, the use of multiple anti-microbial agents increases the probability that the virus or bacteria is chemically weakened so as to be fully eradicated when exposed to UV radiation and/or ultrasound, or vise versa. Moreover, the combination of chemical means with mechanical, light, and sound energy allows for targeting to both maximize their effectiveness against contagions while at the same time reducing the potential for survival/proliferation of resistant strains as well as side-effects for the human user or the accessories being sterilized.

In addition, the inner surface(s) of the tunnels 110 be rifled for the creation of a venturi effect when moving gasses or liquids during the sterilizing process. This inner rifling pattern may further be lined with UV, Ultrasound, laser, hydraulic, and/or pneumatic emitters, e.g., equidistant but close enough to each other, to cover the full surface area of the user's appendages with their emissions. In yet further embodiments.

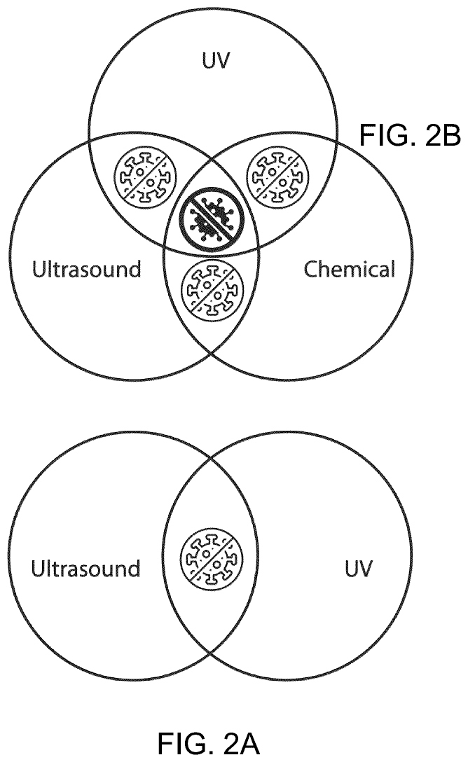

FIGS. 2A and 2B schematically depict the complimentary and synergistic effects of the above described processes. FIG. 2A schematically shows the increased effect on a pathogen when exposed to both UV and Ultrasound components. FIG. 2B schematically shows the increased effect on the pathogen when exposed to two of UV, Ultrasound and chemical components, and the yet further effect when exposed to all three vectors.

Unit 100 also includes an accessory chamber 175, though other embodiments do not include an accessory chamber. The accessory chamber 175 sterilizes jewelry, tools, electronic devices (e.g., cell phones) or other handheld objects along with or prior to the disinfection of the user, in order to prevent reinfection of the user by the accessories. The accessory chamber 175 uses similar processes to sterilize the accessories and the chambers 110 do for the skin. However, the chamber can be configured to utilize processes and/or different intensities and/or frequencies than the chamber 110 does so as to not damage the accessories, especially electronic devices, such as smartphone and/or electronic keys. For example, certain chemical antimicrobials, while safe for sterilizing human skin, can be caustic and damaging to electronics. Conversely, because the processes used in the accessory chamber 175 need not be safe for human skin, the processes for the accessory chamber 175 may use intensities, frequencies and durations that are not safe for human skin, in order more effectively sterilize the accessories. As but one example, the level of NO₂ in the accessory chamber 175 may exceed what is safe for humans, so long as measures are taken so that the user or nearby persons are exposed to the NO₂.

The accessory chamber 175 may be constructed in a similar manner as is the chamber 110. The inner volumetric size of the chamber may be sized as needed to accommodate the accessories expected to be disinfected thereby, which can depend on user(s) or the commercial or societal sector that the unit 100 is configured to serve. Thus, while an accessory chamber intended to sterilize small personal items such as jewelry or cell phones may be rather small, an accessory chamber intended to sterilize items such as large tools may be much larger. Accordingly, the size of the accessory chamber 175 shown in FIG. 1A, both in its absolute size and size relative to the chambers 110, should be understood to be exemplary and schematic, and not defining. In at least some embodiments, the accessory chamber can be used independently of the portions that disinfect humans, if so desired, and sterilize only the accessories.

As noted above, the unit 100 has a control system to control and operate the unit. The unit 100 has a user interface 115. The interface 115 may be equipped with a card scanner, fingerprint reader, facial recognition scanner, or other forms of identification (ID) scanners to identify the user or operation/maintenance personnel, e.g., via RFID, badge ID, bar code or QR code. One use of such user information would be utilized by the controller to adjust the height of the unit, as discussed above, or to set the duration and type of sterilization cycle the user requires. Alternately, said information may be input manually into the controller by typing in parameters or user identification, or stored in a GME or SaaS database, and accessed via the local or wide area network.

The unit may include sensors for control and data gathering. This can include, for example, sensors for sensing the pressures and temperatures of the gases and liquids used, which can affect not only the sanitizing process but also the safety of the user. Such sensors can include, by way of example only, near, mid, and far wave infrared sensors. It can also include sensors for sensing the cuffing pressure, for example, to assure a sealing contact with the skin without pain or injury to the user.

The tunnels 110 may also contain sensors to aid in the operational sequence. These may include LIDAR as a proximity sensor and as a foreign object detector (or another foreign object detector) to detect metals such as jewelry, so as to alert the user to remove the metal from the chamber 110 and, if so equipped, sterilize it in the accessory chamber 175 if desired. The sensor array may also include Ultrasonic distance measurement as a proximity switch or to aid optimal hand/forearm positioning in the tunnels 110. The tunnel 110 can also include buttons (e.g., mechanical, capacitive, etc.) for operating closure/opening of the seal 140 and/or beginning the sanitation cycle.

The above merely constitute examples of sensors and control systems that may be utilized with the invention, and are not limiting. Those of ordinary skill in the art should understand that other sensors and controls may be utilized with the invention, and what those sensors and control may be.

It is also contemplated that the unit 100 (or other sterilization units) may be networkable and part of a local network and/or connected to a cloud network which can provide the unit with real-time updates and/or alter its configuration to target real-time contagion outbreaks identified by a server dedicated for such a task. To do so, the unit's control hardware may be equipped with additional sensors, such as biosensors, e.g., pathogen sensors, rapid analytical biosensors, waveguide-based biosensors, electronic-nose devices, and/or loop-mediated isothermal amplification (LAMP), which detects pathogens by amplifying their DNA, and other biosensors that are known or will become known to those of ordinary skill in the art, in order to collect local information about the locally occurring contagions it encounters and send said information back to the server, in order to inform the decisions made by the service as to how to configure the other units in the local network to respond to viral outbreaks as they happen. The collected information may also be fed back to a local or wide area network, where said information about emerging contagions can be processed and new sterilization configurations altered and fed back to the devices on the network. These decisions may be made by exchanging viral outbreak and seasonal infection data with organizations such as the WHO and the CDC. The units may be programmable to alter the emitted frequencies, intensities, and chemical mixes locally or globally.

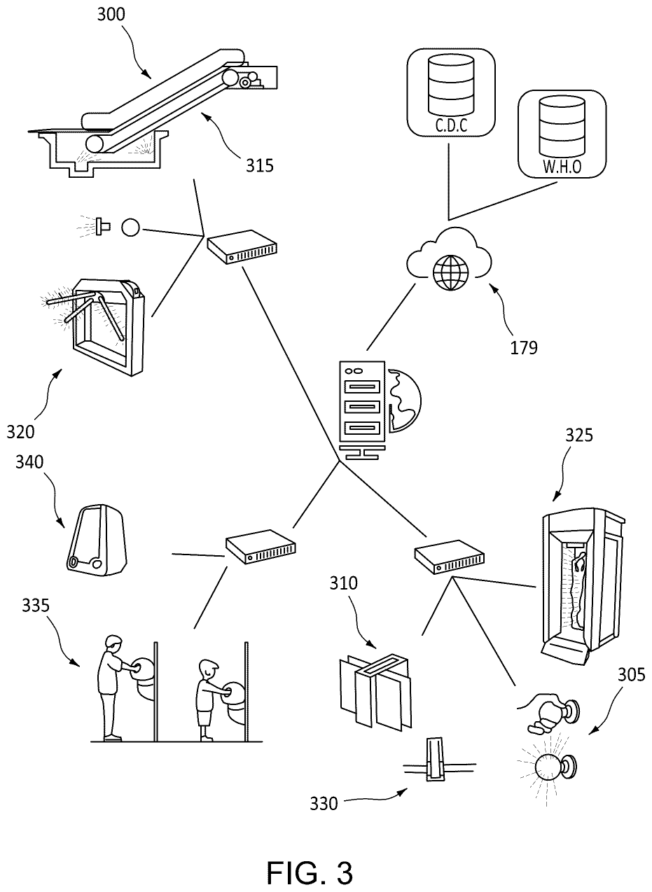

In addition, with reference to FIG. 3, it is contemplated that disinfection units can be part of a network 300 of multi-process devices and architectural elements. Such devices and elements may include doorknobs 305 and emergency exit push-bars, interior office walls 310, elevator and escalator 315 components, turnstiles 320 and swing door assemblies, dedicated full-body sterilization tunnels 325, metal detector tunnels retrofitted and upgraded to include sterilizing facilities, toilet seats, faucet fixtures, towel dispensers, and bathroom stall walls that vibrate at the targeted ultrasound frequencies and can be showered with the targeted frequency of UV light and chemical agents, subway car seating and railings, stadium and theater venue seating and railings, taxicab door handles, seating, and other hardware 330, hospital and office tabletops, computer keyboards and mouse, ATM buttons, screens, and interface components, and other body part disinfection systems 335, 340. Accordingly, disinfection units as well as disinfecting connected devices (such as door knobs, handrails, escalators, elevators, and a multiplicity of other networked devices) can be informed and coordinated so as to act in unison as a complete viral-vector neutralization system. The system can help prevent infection and, importantly, reinfection of a user's hands after completing a sanitizing cycle due to contact with doorknobs, hand railings and other objects are primary vectors in a contagion outbreak.

Locally, both private devices (businesses, doctor's offices, etc.) and public/quasi-public devices (hospitals, schools, government offices, highly-populated public spaces) that have been strategically deployed to achieve a high coverage percentage can be used in such networks. Via pathogen sensing capabilities, the individual devices can detect pathogens, and in at least some embodiments, the viral and bacterial loads of individuals, groups and communities. This information can be communicated to local, regional national and global entities for detection and tracking. For example, on the local level, data can be transmitted to local hospitals, police departments, fire departments, and local first responder government agencies. The data could also be sent to state and/or national agencies, e.g., the CDC, FDA, NIH, etc., and/or global agencies such as the WHO, European Medicines Agency (EMA), etc. For certain pathogens that may be associated with terrorist activity or biological warfare, such as anthrax or hemorrhagic fever, appropriate agencies such as the FBI could be notified.

It should be understood by those or ordinary skill in the art that, for detecting pathogens, what suitable biosensor(s) can be used to detect the target pathogen(s). One of ordinary skill in the art should understand what biosensors are currently known, and will understand what biosensors later become known, that can by utilized in the invention.

In at least some embodiments, the notifications can be made on a “push” basis. For example, individual devices could collect information and transmit it at a predetermined interval, e.g., daily, weekly, etc. In at least some embodiments, the individual units can programmed to transmit notifications) when a certain pathogen is detected, when a certain level (number) of pathogen is detected, and/or when such is detected at certain frequency (e.g., a threshold number of individuals or portion of the population). In least some embodiments, authorized agencies, such as the CDC, could update the units' reporting criteria.

For example, if the Department of Homeland Security (DHS) received a credible terrorist threat of weaponized small pox, DHS could update units to recognize smallpox (if within the hardware capabilities of the machine) and report any detection of the virus. As another example, upon discovery of a new COVID-19 variant, a unit can be programmed to report detection of same to provide an early indication of spread.

In other embodiments, the notification can alternatively or in addition be made on a “pull” basis. For example, an individual unit can report collected data when requested, e.g., by the FDA. Such embodiments permit health and government agencies to obtain data when desired, and not wait for a routine or triggered reporting from the unit.

Once an outbreak has been detected, the network of units could in a similar manner be utilized to track the outbreak. Such embodiments provide advantages over commonly-utilized techniques. For example, during the COVID pandemic, tracking often depended upon reporting by medical practitioners who either diagnosed patients with COVID or reported COVID test administration results, or self-reporting by individuals from their homes. This, however, depended in large part on persons becoming ill (as opposed to being symptomless or near-symptomless), and such persons going to a medical facility or self-reporting, and thus likely failed to capture a significant number of infected persons.

Other types of detection, such as sewage monitoring, has been attributed with greater accuracy in reporting infection rates. However, while such techniques could detect overall rates within a population, it was much less useful in determining infected sub-groups or individuals within the population. In contrast, with the invention, because detection can be attributed to a particular device, whose location is known, particular sub-group or at least locations (for example, a particular school) can be identified and targeted for remediation, e.g., testing, treatment, quarantine, etc.). Yet further, where the individuals using a machine are know, such as by use of an ID card, infected individuals can be identified, notified, and treated as necessary.

In addition, the reporting discussed above was generally delayed, such that data received by authorities could be at least days old. With the present invention, reporting can be essentially instantaneous upon detection.

In yet further aspects, the networked devices can be utilized to monitor use and/or compliance with mitigation protocols. For example, the use rates (or lack thereof) of the disclosed disinfection machines can indicate spread risk levels and help health authorities determine what actions to take within that community. A measured low use-rate, for instance, could help lead authorities to devote additional resources to that community, including, by way of example, providing additional educational outreach, setting up additional testing sites, or, if warranted, pre-staging medical treatment resources if an increase in ill patients is expected.

Turning back to FIGS. 1A-1B, unit 100 may be configured, for example, to use as surgical disinfection device. Surgical theaters demand that anyone participating thoroughly scrub their hands and forearms. More specifically, the international standard is for the user to maintain their hands pointing upwards until the moment the surgeon is fitted with surgical gloves so any liquid residue dripping off the user will drip down and away from the hands, preventing contamination on unsanitized skin from transferring to sanitized skin. The same “upward” requirement applies to sanitization using the unit 100. As can be seen from FIGS. 1A and 1B, the unit 100 and its tunnels are configured so that the user places his arms 10 into the chambers 110 pointing upwards.

In at least one embodiment for surgical use, unit 100 utilizes ultraviolet light spectrums UV-A, UV-B, but also UV-C at 254 nm and 222 nm, as UV-C, in combination with ultrasound between about 20 and 100 Mhz, and disinfecting gases, e.g., ozone or nitric oxide at a concentration of about 160 ppm, at overpressure to force the gases into crevices of the user's body part and of the inner tunnel walls of the tunnels 110, as well as one or more fluid antimicrobial agents such as ST37 for a user defined or network programmable duration between about 3 and about 8 seconds. One of ordinary skill in the art should understand what gases and fluid agents, alone or in combination, to utilize to target the pathogen the societal sector or industry seeks to target. For example, agents generally-referred to as “sanitizers,” such as ethyl alcohol and isopropyl alcohol, are considered relatively effective against bacteria, but less so against fungi. Thus, where the sector is seeking general sanitation against bacteria, e.g., food service, an alcohol-based agent might be suitable. Conversely, in the situation of an invasive fungi within community, a different agent might be used, as those skilled in the art should appreciate.

The unit 100 is connectable to a local, wide area network, and/or cloud server that can update the configuration of the unit 100 based on emerging contagion data sourced from agencies such as the Center for Disease Control and the World Health Organization. The unit's configuration and user data may be stored in a GME or SaaS database servers.

The unit may be equipped with a variety of sensors including LIDAR and Ultraviolet sensors for foreign metal object detection, Ultrasonic proximity sensors, Infrared in near, mid, and far wave detectors, and air pressure sensors, such as discussed above. These sensors systems can be utilized to control the sanitizing sequence and to track system performance, system status, and usage. This sensor array can also act as a monitoring system to identify maintenance or service needs to a local or web enabled notification or scheduling system.

As discussed above, the unit 100 is height adjustable. The unit 100 can, in various embodiments, determine or obtain the user's height automatically, e.g., using LIDAR sensors, or via user entry using the interface or automatically obtained from the server database via the use of fingerprint scanner, facial recognition, an ID card scanner, FOB, swipe, or other ID.

As also discussed above, the unit 100 includes an accessory chamber 175 for sterilizing jewelry, electronics, etc. of the surgical personnel. In the modern surgical theater, surgical personnel require electronics such as cell phone and tablets at the operating table. By sanitizing such using the accessory chamber 175, reinfection of the personnel is avoided. Conversely, for non-essential items such as jewelry, such can remain (sterile) in the accessory chamber 175 until the surgical procedure is completed.

Accordingly, at least some such systems for surgical use may operate in the following exemplary manner:
(a) The device may be continually checking with a local or wide area network server for updates and process configuration changes as triggered by emerging contagion threats identified by hospitals, labs, and agencies such as the Center for Disease Control (CDC) and the World Health Organization (WHO).
(b) User specific parameters are called up by the scanning of a hospital employee's credentials, in order to properly adjust the unit to the specific user's height requirements, arm length and circumference. The user's credentials would be utilized as well to set personal temperature preferences and company sanitation requirements and standards that may include updated configurations based on emerging viral threat information sourced from the cloud server connected to the CDC and WHO.
(c) User would be invited to place hand-held devices or jewelry in an optional accessory chamber, which will be subject to similarly configured sterilizing processes or processes specifically tailored to the types of devices a medical industry specific configuration would call for.
(d) The machinery automatically adjusts to the user's height using either the optional wall-mounted railings or the specially designed floor mount stand that incorporates automatic height adjustment tracks and motors. The unit's internal mechanism would alter the tunnel circumference to adjust for the user's forearm length, hand size, and forearm circumference via internal mechanical scissor type, rotary expandable, or fan type collapsible tunnel structure.
(e) The user places his/her arms in the machine with the hands pointed upwards. The self-sealing apertures close tightly enough around the user's forearms to create a hermetic seal. These cuffing seals may be inflatable pneumatic cuffs similar to those utilized in blood pressure testing equipment aided by elastic neoprene rings.
(f) The UV-A (UV-B and UV-C as alternatives) emitters turn on and optionally stay on throughout the entire cycle/process until the user has left and the unit has finalized is post-use self-cleaning routine.
(g) A metal detecting phase that utilizes the internal LIDAR and Ultrasonic sensors would warn the user of any jewelry that may interfere or be damaged by the vigorous automated process via specially designed sounds, flashing lights, and messages on the touchscreen. This would provide the user with the opportunity to remove his appendages from the unit and remove the jewelry and place them in the accessory chamber prior to restarting the process.
(h) The process of disinfection would begin, and optionally this process would include the accessory chamber if so determined via the process in Step No. 7.
(i) Optionally, gasses including, but not limited to ozone, nitrous oxide, and some combination of fluid antimicrobial agents such as ST37 would be released by the internal spigots powered by the internal pneumatic and hydraulic pumps at system configurable pressures and varying percentages. The spigots would be evenly distributed along the inner surface of the tunnel chambers at interval distances frequent enough to cover the entire skin surface of the user's appendages, and following the rifling pattern utilized to distribute said gasses in a high-speed turbulent manner as to disrupt and dislodge dirt and dead skin. This hydraulic and pneumatic decontamination phase would last for 2-30 seconds of variable and system configurable duration. Throughout this process both of the sealed tunnel chambers that hold the user's appendages would be filled, as well as potentially separate/accessory chambers that house apparatuses that require a pre-cleaning.
(j) The Ultrasound portion of this automated sequence would commence by emitting ultrasound waves at system configurable rates and intervals that cycle through the about 20 Mhz to 100 Mhz range. The sonic portion of the process benefits greatly if applied through liquid or gas. The rates and intervals would be programmable to target pathogens and contagions identified by the hospital staff based on CDC and WHO information provided by the network servers.
(k) For a second time, A hydraulic portion of the automated sequence would open various spigots, nozzles, and other pressure washing components which would be made out of anti-microbial materials such as, but not limited to, stainless steel and brass. This hydraulic portion of the automated sequence would again forcefully distribute the antimicrobial fluids and gasses across the full surface area of the user's appendages until the container is flooded. A determination of volume and intensity would be determined by the reprogrammable system, and monitored and metered by the array of internal sensors.
(l) Optionally, a repeat of step 5 and 7 through 10 could be configured when a second phase of disinfection is determined to be desirable by the hospital staff based on information about current or emerging viral and contagion threats gathered via the network as described in Step (a).
(m) A pneumatic drying stage with heated and pressurized hot ionized air directed at the user's appendages.
(n) Once the user's appendages are determined to be suitably dry by the internal sensors, the unit would suction out of the gasses and liquids utilizing its internal pumps and replace with regular air.
(o) An additional sequence of UV-C and Ultrasound bursts can optionally be added here if the specific medical or scientific configuration calls for it based on the information gathered from the network connection to the CDC and WHO.
(p) An image or scan of the hands and forearms is taken using X, Y, and Z Cartesian coordinates to measure before and after bacterial count utilizing biosensors that can scan for and detect the presence of pathogens, contagions, or viruses.
(q) The UV-A (UV-B and UV-C as alternatives) emitters turn off.
(r) The unit would release the self-sealing apertures to allow the user to withdraw. Simultaneously, the unit would open the chamber to the accessory bin, and allow the user to withdraw their hand-held devices of jewelry if said configuration calls for this option.
(s) The self-sealing apertures would completely close in order to conduct a self-cleaning and self-drying sequence that makes the unit aseptic and ready for the next user. This drying sequence would employ the increasing of internal pressure 3 to 5 ATM (3 to 5 times atmospheric pressure) simultaneously irradiating of heat energy in order to push out any lingering fluids that may have remained in the unit's various chambers post-cycle.
(t) The user-specific process and parameters would be logged via the network onto SaaS or GME database servers so they would be available for auditing purposes in the future, and as a human resources tool. The unit would upload statistical information collected about the pathogens it encountered in the user session to the local server and/or to the global network that processes this information in conjunction with the WHO or CDC.
(u) The casing of the unit, which may or may not be made out of anti-microbial materials, such as copper or specially engineered plastics, can be subjected to the same ultrasound repertoire in order to sanitize its exterior surface. The use of UV can also be utilized by strategically placed emitters and sensors, that first determine if a human is in proximity, and then shower the exterior of the unit with UV light once the room is cleared.
(v) Unit goes into energy-conservation mode by turning all light and sound emitters off, and displaying the specially designed message on the touchscreen. The unit would await next user, and be called back into action by detection via an external LIDAR or other proximity sensor, or simply by user entry into the touchscreen, utilizing fingerprint, card, or fob swipe to commence the cycle.

It should be noted that a system that satisfies the technical needs of a surgical environment presents a unique set of ergonomic hurdles. The machine must mimic the physical requirements and prescribed postures of pre-surgical routines, including engaging with the user while the user has their hands pointed upward. Several aspects of the unit 100 achieve this. In one aspect, as seen in FIG. 1B, for example, the side of the enclosure 105 facing the user is shaped so as to create a stance relative to the enclosure 105 while allowing the user to insert their arms 10 into the tunnels 110 in an “arms upward” position that is typical of a traditional pre-surgical scrub stand/position. This includes, as seen in FIGS. 1A and 1B, the upper arms being able to be angled between about −5 to +20 degrees to the horizontal, the lower arms being able to be angled between about 0 to 30 degrees to the vertical. In addition, as seen in FIG. 1A, the two tunnels 110 may be angled away from being parallel to each other in order to create an about 0 to 25 degree angle of the lower arms relative to each other, which is a natural (comfortable) angle for the user. In addition, this positioning permits the upper arms to pass through the openings 135 near perpendicular to the plane of the openings, which helps obtain a secure seal around the arm by the seals 145. Yet further, the openings are placed a lateral distance apart from that permits a natural/comfortable positon for the user to hold their arms laterally apart from each other. In at least some embodiments, the position and angle of the tunnels may be adjustable/user configurable to best accommodate an individual user.

The above-discussed ergonomic strategy is intended to mimic pre-surgical arm postures, and specifically, to interface the unit 100 with the human at specific ranges of angles in order to sustain comfort and a secure hermetical seal throughout the sanitization session. The inventors identified the specific angles shown in FIGS. 1A and 1B as advantageous to do so.

More generally, the inventors have identified the angle of attack of the user's body part to the sanitization unit as a significant factor in the comfort of the user in using the device. The angle of attack is an angle of a body part relative to a reference that the machine requires the user to achieve in order to insert and maintain the body part in the machine.

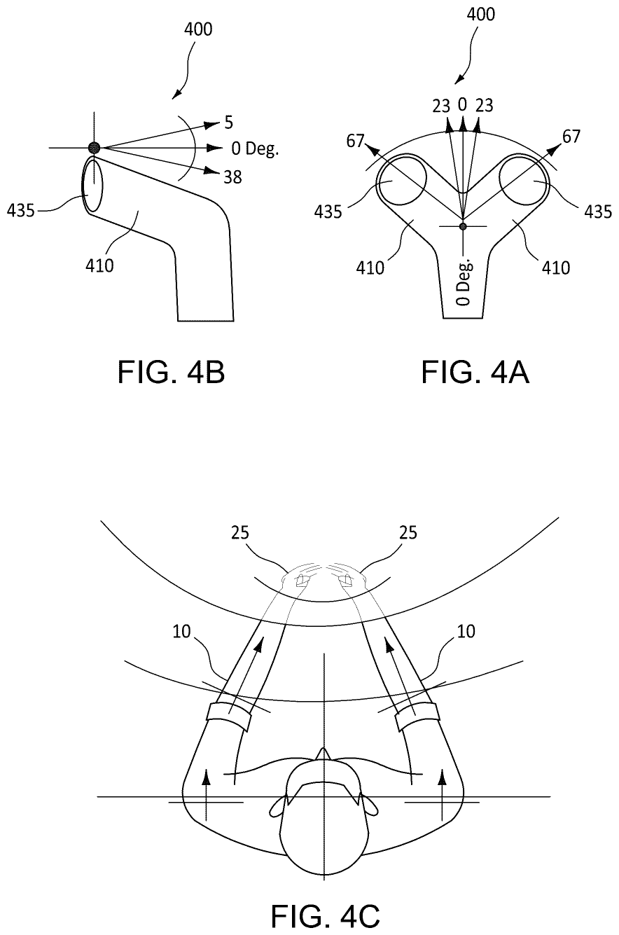

FIGS. 4A and 4B schematically show another disinfection unit 400. Unit 400 is similar to unit 100 described above, and therefore like reference numerals in FIGS. 4A-4B preceded by the numeral “4” instead of the numeral “1” are used to indicate like or similar elements. Unit 400 includes two tunnels 410, each adapted to receive respective wrist areas and hands 25 of the left and right arms 10, respectively. As shown in FIGS. 4A and 4B, each tunnel is configured to allow an angle of attack of the respective arm to be about ±23 degrees from the vertical and about −38 to +5 degrees from the horizontal, with the axes of the tunnels about 67 degrees apart. As seen in FIG. 4C, this permits a wide variety of users to insert their hands into the tunnels 410 in a generally natural/comfortable posture position.

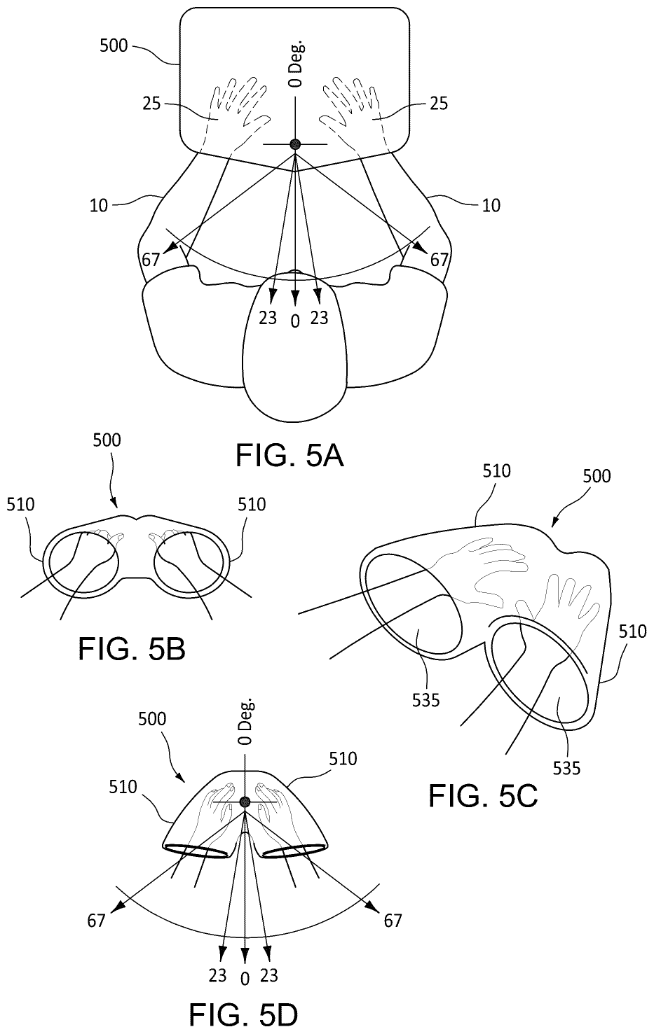

FIGS. 5A-5D schematically show another disinfection unit 500. Unit 500 is similar to units 100 and 400 described above, and therefore like reference numerals in FIGS. 5A-5D preceded by the numeral “5” instead of the numeral “1” or “4” are used to indicate like or similar elements. Unit 500 includes two tunnels 510, each adapted to receive respective wrist areas and hands 25 of the left and right arms 10, respectively. As shown in FIGS. 5A-5D, though the tunnels 510 are design to accept a different part of the body, they are still configured to allow an angle of attack of wrist arm substantially the same as for unit 400 discussed above. Such is also achieved in part by curving/angling the front face of the unit 500 to allow a comfortable angle of attack to the user's torso. Thus, as with unit 400, unit 500 permits a wide variety of users to insert their arms into the tunnels 510 in a generally natural/comfortable posture position.

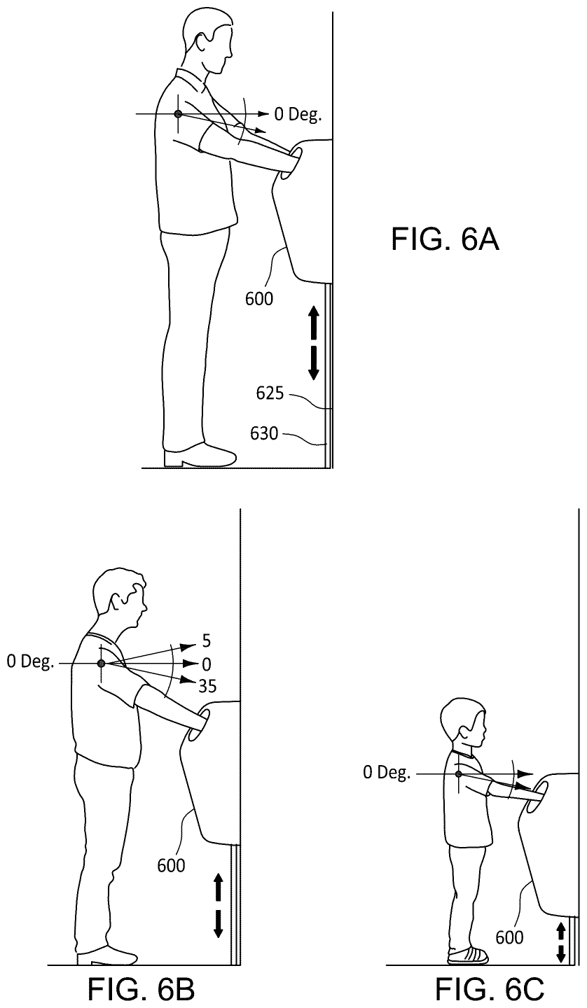

As discussed above, user height plays a significant role in the posture/angle of attack of a user relative to a sanitizing machine. Accordingly, at least some embodiments are height adjustable. FIGS. 6A-6C schematically show a further disinfection unit 600. Unit 600 is similar to units 100, 400 and 500 described above, and therefore like reference numerals in FIGS. 6A-6C preceded by the numeral “6” instead of the numeral “1”, “4” or “5” are used to indicate like or similar elements. As seen in the figures, unit 600 is height-adjustable. This adjustability allows, as also seen in FIGS. 6A-6C, users of significantly different heights adjust the height of the unit 600 on the wall-mounted support 625/rail 630 so as to achieve a natural/conformable angle of attack relative to the unit (e.g., about −35 to +5 degrees relative to the horizontal).

As opposed to the above-discussed unit 100 and surgical embodiments thereof, other embodiments, such as units 400, 500 and 600, disinfect only from the user's wrists to the fingertips. Such embodiments may thus be significantly smaller than unit 100, and, instead of a seal (if present, for example, because the unit uses pressurized gases and liquids) being at or above the elbow, such seal is at the user's wrists. Similar to unit 100, though, the specific combination and utilization of light, sound and chemicals can be tailored to the needs of user or the societal sectors or commercial or industry needs. For example, in a food-related industry that is concerned with the Salmonella virus, the frequency and intensity of the ultrasound energy can be configured to target the specific weaknesses of Salmonella. As another example, in a sector or commercial industry where the MRSA virus is of concern, such as in nursing stations as opposed to pre-surgical rooms, and the frequencies and intensities could be configured via program settings for that virus.

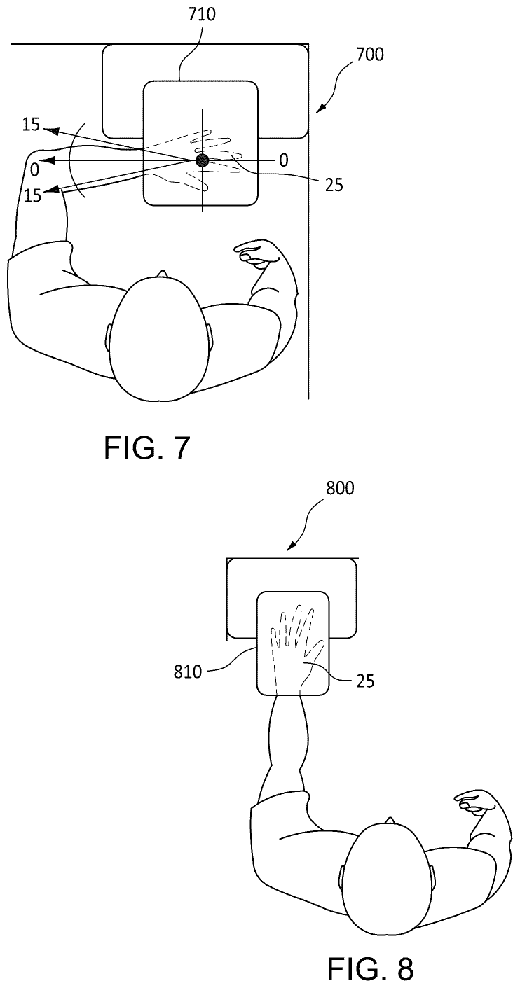

FIGS. 7 and 8 schematically show a further disinfection units 700 and 800. Units 700 and 800 are similar to units 100, 400, 500 and 600 described above, and therefore like reference numerals in FIGS. 7 and 8 preceded by the numeral “7” or “8” instead of the numeral “1”, “4”, “5” or “6” are used to indicate like or similar elements. As seen in FIGS. 7 and 8, units 700 and 800 contain only a single tunnel 710, 810 to receive a wrist/hand of the user. Unit 700 is configured for the user to insert their hand 25 into the tunnel 710 by bending their arm at the elbow. Unit 800 is configured for the user to insert their hand 25 into the tunnel 710 with their arm straight. Yet both units allow a natural/comfortable angle of attack. Of note, tunnel 710 is configured to allow a natural angle of attack with the elbow bent ±15 degrees relative to the forearm being perpendicular to the upper arm.

However, in order for above-discussed disinfection units to permit a comfortable angle of attack for uses of different heights and sizes, the openings may be relatively large. Such may requires a user to hold their arm/hand in space for perhaps a significant period of time. Especially for those that are physically less able, this may be tiring or challenging.

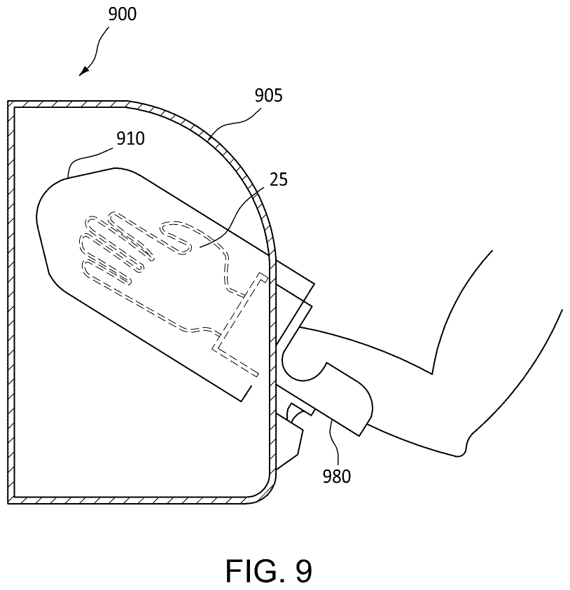

FIG. 9 schematically shows a further disinfection units 900. Unit 900 is similar to units 100, 400, 500, 600, 700 and 800 described above, and therefore like reference numerals in FIG. 9 preceded by the numeral “9” instead of the numeral “1”, “4”, “5”, “6”, “7” or “8” are used to indicate like or similar elements. As seen in FIG. 9, unit 900 contains a tunnel 910 for receiving a user's hand 25. In addition, until 900 includes support 980 against which the user may rest their wrist or arm, to lessen the difficulty or tiring of holding the arm/hand in space. In at least some embodiments the support 980 is adjustable, e.g., via gimbals, so that the user can obtain a comfortable angle of attack while resting on the support 980.

As discussed above, embodiments used for other than surgical purposes may use a different process than one used for surgical purposes. At least some such systems may operate in the following exemplary manner:
(a) The device may be continually checking with a local or wide area network server for updates and process configuration changes as triggered by emerging contagion threats identified by hospitals, labs, and agencies such as the Center for Disease Control and the World Health Organization.
(b) User specific parameters are called up by the scanning of employee's credentials, specifically, but not limited to temperature and height requirements, or if in a commercial retail environment, simply log the user's information for statistical use.
(c) Of importance would be for the introductory phase to determine if the user is handicapped, and only requires one chamber for a single hand to be sanitized. The device would know to seal the second device for single-handed users via manual entry into the touchscreen, or by accessing user information from the SaaS or GME servers through the network as determined by User ID in the form of fingerprint readers, card or fob swipes.
(d) User would be invited to place hand-held devices or jewelry in an optional accessory chamber by the touchscreen and specially designed sounds and lighting. The accessory chamber will be subject to similarly configured sterilizing processes or processes specifically tailored to the types of devices an industry specific configuration would call for, differentiating between the types of viruses common to the industry, or as specified via the network connection to information as provided by agencies such as the CDC or WHO.
(e) The machinery automatically adjusts to the user's height via either the optional floor stand or wall mount with tracks and motors. Optionally, this embodiment would alter the tunnel circumference and length to the hand size, and forearm circumference utilizing a collapsible tunnel structure that may comprise of mechanical scissor-type, fan-type, or rotary expandable structure system.
(f) A metal detecting phase utilizing the internal LIDAR, Ultrasonic, or magnetic-types metal detectors would warn the user of any jewelry that may interfere or be damaged by the vigorous automated process. The touchscreen would display a specially choreographed message along with sounds and lighting effects to provide the user with the opportunity to remove his appendages from the unit and place the jewelry the accessory chamber prior to starting.
(g) The UV-A (UV-B and UV-C as alternatives) emitters turn on and optionally stay on throughout the entire cycle/process until the user has left and the unit has finalized is post-use self-cleaning routine.
(h) The process of disinfection would begin, and this process would include the accessory chamber if so called for.
(i) Optionally, ozone, nitrous oxide, and some combination of fluid antimicrobial agents such as ST37 would be emitted from the array of spigots lining the inside surface of the tunnel chambers in varying pressures and varying percentages. These mixtures would be released for a variable duration of 2 to 30 seconds, thus filling the various sealed chambers that hold the user's appendages as well as potentially separate/accessory chambers that house apparatuses that require pre-cleaning.
(j) In the event the configuration does not use any liquids, the gas may optionally be isolated from the outside environment by utilizing a wall of high-pressure air at the entry point of the tunnel orifices.
(k) The Ultrasound portion of this automated sequence would commence by emitting ultrasound frequencies in a variable sequence of wavelengths ranging from about 20 Mhz to 100 Mhz. The variable sequence would be tailored for the industry to target the pathogens common to the industry or societal sector, or as dictated by updates to the cloud server based on information gathered from agencies such as the CDC or the WHO.
(l) Optionally, A second hydraulic portion of the automated sequence would open the various spigots, nozzles, and other pressure washing components lining the inside surface of the tunnel chamber walls. These spigots may be made out of anti-microbial materials such as, but not limited to, stainless steel, silver, brass and anti-microbial polymers. This optional hydraulic portion of the automated sequence would forcefully distribute antimicrobial fluids across the full surface area of the user's appendages until the container is flooded. The unit would determine flow and duration via its array of internal sensors, and adjust accordingly to what the industry calls for in order to combat known pathogens to be common to the industry or societal sector.
(m) An optional pneumatic drying stage with heated and pressurized air directed at the user's appendages if the configuration that calls for the use of liquids or gasses. The unit would detect lingering amounts of fluids via its internal array of moisture sensors and evacuate them via pneumatic pressure accordingly.
(n) Once the user's appendage(s) are suitably dry as determined by the internal sensors and compared to atmospheric humidity of the particular geography the unit is installed in, the unit would suction out of the gasses and replace with regular air via use of its internal vacuum pumps. In a configuration that provides a hermetic seal with the cuffing mechanism, the unit may optionally push out the lingering gasses and liquids by creating 3 to 5 times atmospheric pressure inside of the chambers utilizing its pneumatic pump.
(o) An optional battery of UV-C and Ultrasound bursts can optionally be added here if the specific industry's configuration calls for it, and as determined through the network connection to local or cloud servers sourcing information from agencies such as the WHO or the CDC.
(p) An image or scan of the hands and forearms is taken using X, Y, and Z Cartesian coordinates to measure before and after bacterial count. Sensors capable of detecting contagions would be employed.
(q) The UV-A (UV-B and UV-C as alternatives) emitters turn off.
(r) The unit would release the self-sealing apertures if so employed in order to allow the user to withdraw. Simultaneously, the unit would open the chamber to the accessory bin if said configuration calls for this option.
(s) If the embodiment employs pressurized gasses and liquids, the self-sealing apertures would completely close in order to conduct a self-cleaning and self-drying sequence that makes the unit aseptic and ready for the next user to engage without fear of contamination from previous use. This drying sequence would employ the increasing of several atmospheres and irradiating of heat energy intended to evacuate any lingering fluids that may otherwise collect in the unit.
(t) The user-specific process and parameters would be logged so they would be available for auditing purposes in the future, and optionally as a human resources tool. The unit would upload statistical information collected about the pathogens it encountered in the user session to the local server and/or to the global network that processes this information in conjunction with the WHO or CDC.
(u) The casing of the unit, which may or may not be made out of anti-microbial materials, such as copper or specially engineered plastics, can be subjected to the same ultrasound repertoire in order to sanitize its exterior surface. The use of UV can also be utilized by strategically placed emitters and sensors, that first determine if a human is in proximity, and then shower the exterior of the unit with UV light once the room is cleared.
(v) Unit goes into energy-conservation mode by turning off all pumps, UV and Ultrasound emitters, and awaits the next user. The unit would be triggered to commence a new cycle when it detects a new user via LIDAR or proximity sensor, via manual input into the touch screen, or by user ID utilizing fingerprint scanner, facial recognition scanner, or a card or fob swipe.

Yet further embodiments may use the same or similar array of technologies to address the needs of certain persons or groups. For example, extensive research by the inventors has discovered that cleaning/disinfection/sterilization is more effective if the chambers are be sealed off during the process, e.g., sealing around the user's body part extending through the chamber opening(s). Further, the inventors have discovered that, regardless of whether the chambers are sealed, the process is more effective if the user participates/interfaces with the machinery in a calm and orderly manner. However, this can pose challenges for certain persons or groups, such as children, those with lower cognitive or emotion maturity/development, those with certain mental conditions such as, by way of example only, anxiety or Cleithrophobia, or those who may be physically less able.

In a first aspect there may be challenges physically interfacing with the device. As discussed above, one goal is to provide a suitable angle of engagement, along with suitable sealing. The inventors' research revealed that may be more difficult to accomplish when physical attributes (e.g., height) and able-ness are variable and/or influx. An example would be school children, who may vary in age, height and size generally. In that and similar instances, height and hand size parameters may be manually set by the device operators, e.g. hospital or school staff, to meet the age, body size and physical capabilities. In other embodiments, the unit's external LIDAR sensors may scan for user height and body shape and adjust the height via the unit's height adjustment mechanisms, which may for example, be built into a floor-stand or wall mount structure for the unit. As another example, at least some embodiments use sound and assisted engagement for those unable to read or understand text in a digital display.

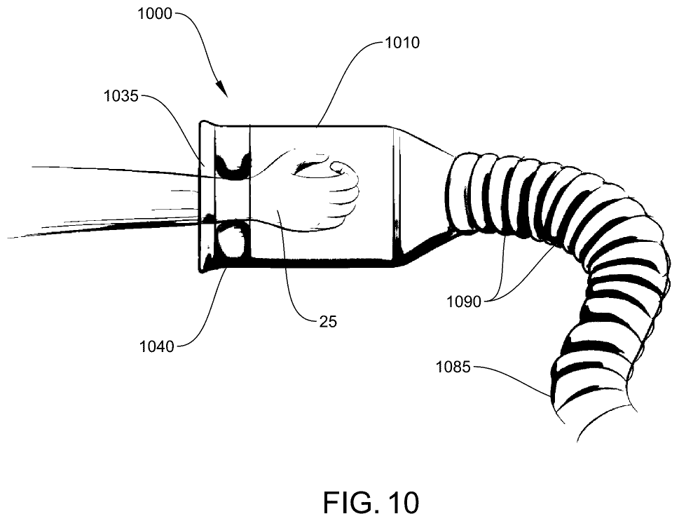

FIG. 10 schematically shows another embodiment of a disinfection unit 1000. Unit 1000 is similar to units 100, 400, 500, 600, 700, 800 and 900 described above, and therefore like reference numerals in FIG. 10 preceded by the numeral “10” instead of the numeral “1”, “4”, “5”, “6”, “7”, “8” or “9” are used to indicate like or similar elements. As shown in FIG. 10, the unit 1000 has an articulatable arm 1085 to which the tunnel 1010 is attached and by which the tunnel is supported. The arm 1085 includes a plurality of bendable/expandable/compressible pleats or bellows 1090 that can be bent, extended and/or compressed so that the tunnel 1010 can be positioned at a multitude of positions (vertically and horizontally) and angles within the range of adjustability of the arm 1085. In such manner, the tunnel can be positioned so as to provide the user with a comfortable position and angle of attack of the body part. This embodiment may be particularly advantageous for users who are physically less able, whereby the chamber 1010 can be positioned to accommodate conform to the user (e.g., height, girth, and standing/sitting posture, physical limitations/injuries), rather than the user positioning/conforming themselves to accommodate the positon and configuration of the unit, particularly the chamber and openings thereto.

A second aspect is the psychological willingness and ability to effectively interface with the machine. Anxiety, fear or the like may impede a user from inserting a body part into an unfamiliar device and/or then allowing the unit to temporarily hold them and immobilize part of them during the process. What the inventors have discovered, though, are ergonomic strategies that tailor not just the functionality of the device, the nature of the interface experience, including, for example, sights, sounds and touch(es). By making the machine (more) aesthetically appealing and less intimidating, a user, e.g., the youth or mentally challenged, may be less anxious and fearful and more willing and trusting of the temporary engagement, including immobilization of appendages.

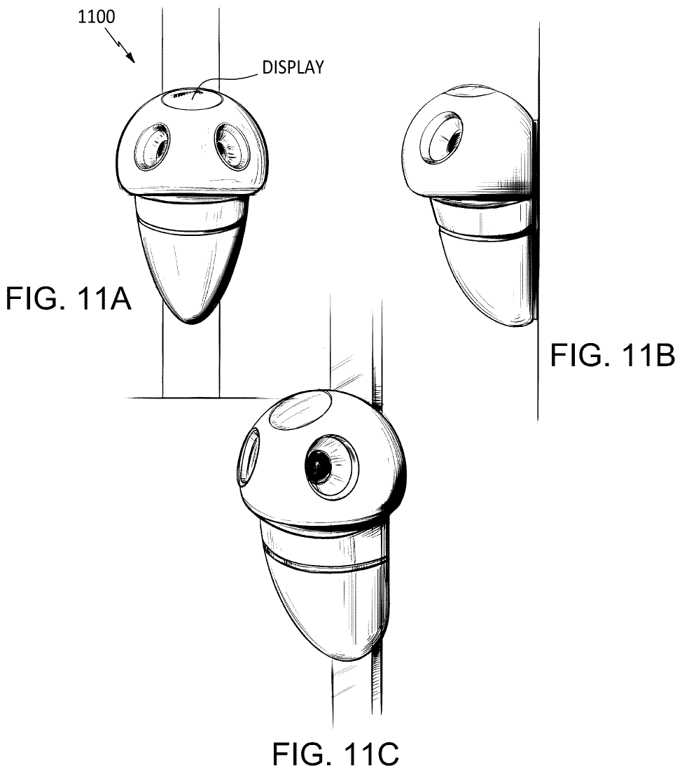

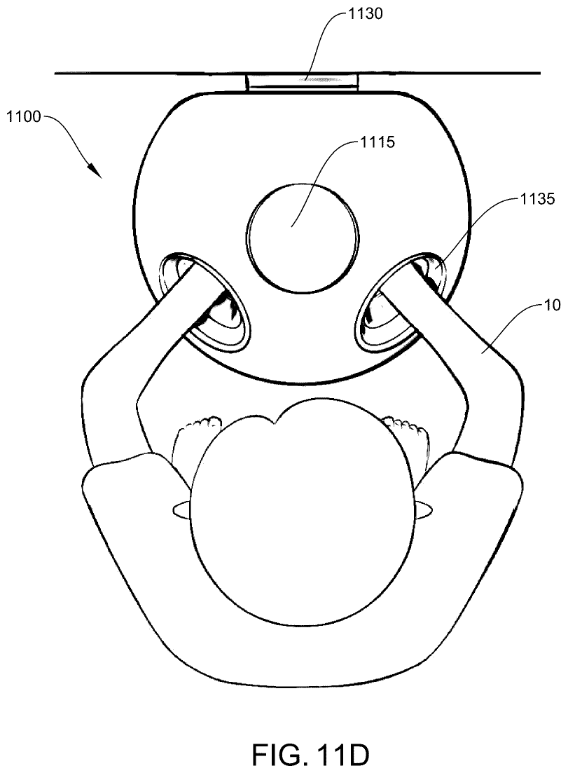

FIGS. 11A-11D shows an embodiment of a disinfection unit 1100 that may provide an enhanced or less stressful experience for certain users, such as, but not limited to, children, those who are less mature, and/or mentally less able. Unit 1100 is similar to units 100, 400, 500, 600, 700, 800, 900 and 1000 described above, and therefore like reference numerals in FIGS. 11A-11B preceded by the numeral “11” instead of the numeral “1”, “4”, “5”, “6”, “7”, “8”, “9” or “10” are used to indicate like or similar elements. Unit 1100, however, differs from other embodiment in various respects. One such difference is the overall size of the unit, which is smaller to accommodate the small size of children as compare to adults. The smaller size is also intended to be less intimidating than “full-size” units for adults. Further, the overall appearance is intended lessen any intimidation or fear by the user. Rather, the appearance is meant to be more aesthetically pleasing to younger or less mature users.

At least some such systems may operate in the following exemplary manner:
(a) The device may periodically or continually check with a local or wide area network server for updates and process configuration changes as triggered by emerging contagion threats identified by hospitals, labs, and agencies such as the Center for Disease Control and the World Health Organization, or updated industry standards or recommendations.
(b) The machine can be programmed to scan the user's body type and adjust its height (in so-equipped embodiments) using an external LIDAR or other sensor, or by voice or keyed input into the machine. In at least some embodiments, the tunnel chamber angle and breadth may be automatically adjusted based on LIDAR scans of body type, utilizing collapsible and expandable scissor-type, fan-type, or rotary-type expansion and contraction tunnel structure.
(c) The design of the interface touchscreen and external speaker system can be programmed to provide soothing and/or friendly voices or sounds intended to put the less able or less emotionally mature more at ease.
(d) Specific parameters can be called up by said scanning of a user's vitals including temperature (e.g., using an infrared thermometer) and height requirements (e.g., LIDAR sensors) that tailor elements such as temperature and intensity of any cleaning/sanitizing methods to help the user remain at case throughout the process.
(c) The previous step could include identifying if the user is handicapped via manual input into the touchscreen or utilizing user ID in the form of fingerprint, facial recognition, or card scanning hardware. The unit could respond, for example, by sealing off selected left or right hand tunnel chambers if, for example, the user lacked a left or right hand, and go unused for the remainder of the cycle. In at least some embodiments, the user could choose to utilize only the accessory chamber, and not participate in the appendage cleaning functionality of the unit(s).
(f) Once the user's body part(s) are inside the (and optionally sealed off), an image or scan may be taken of hands and face, e.g., for record, human resources or customer statistic purposes, utilizing the internal sensors for the hand scan and a camera or LIDAR sensor for the facial recognition.
(g) The self-sealing apertures close tightly enough around the user's body part(s) so as to create a hermetic seal, utilizing a pneumatic bladder cuff system to achieve a partial or fully sealed chamber is achieved. Alternatively, a system that does not employ liquids or highly humid gasses could seal off the tunnel chambers from the outside environment by deploying a high-pressure wall of air at the tunnel entry points.
(h) A metal detecting phase utilizing the internal metal sensors, e.g., a magnetic, Ultrasonic or LIDAR sensor array can warn the user of any jewelry or other objects that may interfere or be damaged by the cleaning process, and provide the user with the opportunity to remove the same from the unit prior to starting the process. Reinstitution of the process may be made in at least some embodiments via manual entry into the touchscreen or via proximity sensors (e.g., ultrasonic, infrared, laser, etc.) inside the tunnel chambers that detect that the user's body part(s) are back inside of the tunnels.
(i) The UV-A (UV-B and UV-C as alternatives) emitters turn on and optionally stay on throughout the entire cycle/process cycling through 254 nm and 222 nm frequencies at variable intervals and durations as determined by the local or cloud based databases, or as manually configured, e.g., by hospital or school staff.
(j) If the configuration calls for it, gasses of varying pressures and varying percentages [what pressures/percentages] of ozone, nitrous oxide, ionized hot air, and a strategic combination [what is a “strategic combination”? provide numbers] of fluid antimicrobial agents, such as ST37 [and what else? How is the combination determined/implemented?], are released for a variable duration [what durations, i.e., the seconds minutes, and how are they variable?], thus filling the chambers that hold the user's body parts as well as any separate (e.g., accessory) chambers that house apparatuses that require a pre-cleaning. (See chemical component section) [what does this mean? What chambers? What is “pre-cleaning”? How is it determined whether pre-cleaning is required. What is “chemical component section?”]
(k) An Ultrasound portion then commences by ultrasonic emitters in the chambers emitting ultrasound waves between about 20 Mhz and 100 Mhz at varying durations and intervals [provide more details/numbers of the “durations and intervals”], as set, e.g., by the school or hospital staff, or via automated reprogramming based on information gathered through its local or wide area network connection(s).
(l) In at least some embodiments,, gas mixtures of nitrous oxide, ionized hot air, and ST37 can be pressed in at variable pressure [what pressure(s) and how made “variable”?] via the array of spigots lining the interior tunnel walls fluidly connected or connectable to sources of the gas(es). This sequence is followed up by a pressurized influx of hot ionized air to push any lingering gases or liquids. High-speed air velocity would be achieved via pneumatic pressure at 3 to 5 times atmospheric pressure, and, in at least some embodiments, conveyed through a rifling pattern in the tunnels of the inner tunnel chamber walls.
(m) In embodiments having and utilizing an accessory chamber, the previous three steps can carried out in the accessory chamber, including but not necessarily simultaneously. This can be done, for example, if the societal sector or commercial industry configuration of the unit so deems it, or based on the recommendations or updates gathered through the cloud connection to agencies such as the CDC or the WHO.
(n) An image or scan of the body part(s) is taken using X, Y, and Z Cartesian coordinates to measure before and after bacterial count utilizing biosensors or other techniques configured to detect the presence of active pathogens.
(o) The unit releases the self-sealing apertures of the enclosure or instructs the user to withdraw when the apertures are unsealed. The unit may utilize a choreographed message via speakers or on the touchscreen, which may, for example, be personalized for the user (e.g., include the user's name, and may also use choreographed sounds and lighting effects.
(p) The unit would then unlock/open the accessory chamber lid/door and allow the user to retrieve their jewelry or other accessories.
(q) In at least some embodiments, the self-sealing apertures would completely close in order to conduct a self-cleaning and self-drying sequence that makes the unit aseptic and ready for the next user. This drying sequence may employ increasing internal air drying pressure to 3 to 5 times atmospheric pressure, while at the same time providing heat energy, e.g., by heating the incoming air or through heat emitters in the chamber itself.
(r) The UV-A (UV-B and UV-C as alternatives) emitters turn off and return to resting mode, awaiting the next user to arrive.
(s) The user-specific process and parameters may then be logged onto the SaaS or GME databases via the network connection so they would be available for auditing purposes in the future, tracking, and/or as a human resources tool. The unit could upload statistical information collected about the pathogens it encountered in the user session to the local server and/or to the global network that processes this information in conjunction with the WHO or CDC.
(t) The casing of the unit, which may or may not be made out of anti-microbial materials, such as copper or specially engineered plastics, can be subjected to an ultrasound process in order to sanitize its exterior surface. The use of UV can also be utilized (in addition or in alternative) by strategically-placed emitters and sensors, that first determine if a human is in proximity, and when such is confirmed, shower/expose the exterior of the unit with UV.
(u) Unit goes into energy-conservation mode (e.g., sleep mode) by turning off all pumps, UV and Ultrasound emitters, and awaits the next user. The unit would be triggered to commence a new cycle when it detects a new user via LIDAR or other proximity sensor, via manual contact with/input into the touch screen, or by user ID utilizing fingerprint scanner, facial recognition scanner, or a card or fob swipe (e.g., RFID, bar code, chip).

Those of ordinary skill in the art should appreciate, though, that other embodiments may be operated in a different manner.

In some applications, it may be advantageous to, in addition to the above-discussed cleaning/sterilization components/processes, include mechanical scrubbing to mechanically remove or dislodge dirt or contaminants from the skin. Such may be used, for example, for applications where the skin, e.g., the hands, may be heavily soiled or contaminated, so as to require harsher and more stringent removal from the skin. This might be desirable or necessary, by way of example only, in certain industrial or medical applications. Mechanical methods include, but are not limited to, hydraulic pressure, pneumatic pressure, and abrasive frictional contact with the human skin or, as appropriate, a hand-held tool or accessory. The duration and intensity of the mechanical mechanism(s) can be varied and programmable so as to meet the sterilization needs of a particular application. Mechanical methods can be strategically combined with other processes, e.g., chemical, light, and sonic, as part of a targeted and designed battery of sterilizing attacks on offending contaminants and contagions. A virus already weakened by UV, ultrasound, and/or chemical attack is generally more susceptible to mechanical damage during the mechanical phase, and vise versa. Moreover, such embodiments may employ harsher applications of pressurized liquids and gases than others. In such embodiments, scaling-off the outside environment from the process via, e.g.,, a cuffed seal around the user's appendages, may be desirable.

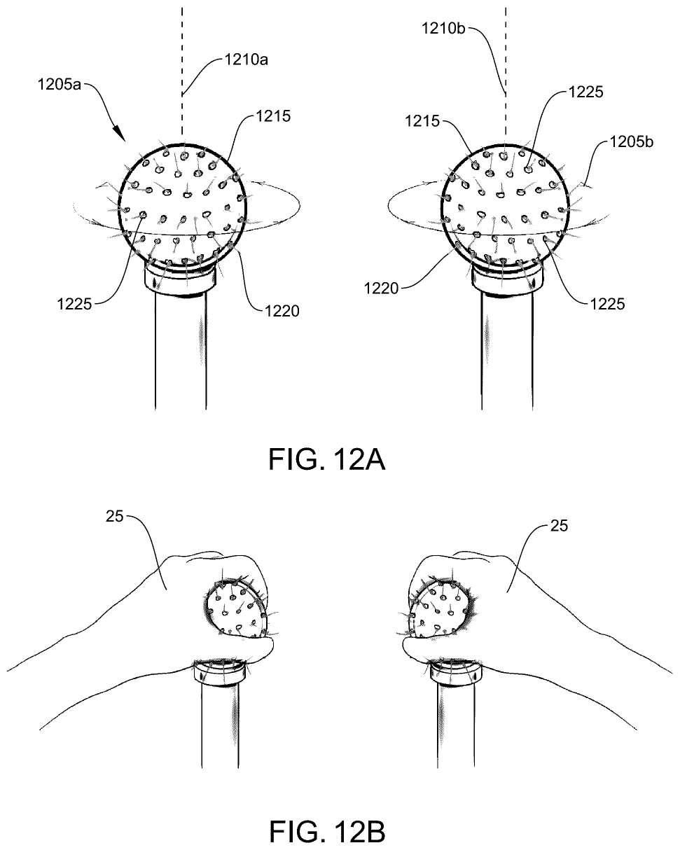

In the embodiment of FIG. 12A, first and second spherical scrubbers 1205a, 1205b are rotatable around respective axes 1210a, 1210b. The scrubbers 1205a, 1205b can be rotated by any suitable mechanism, as one of ordinary skill in the art will understand. They may, for example, be rotated mechanically, electromechanically, e.g., by one or more motors (not shown), or by internal hydraulic or pneumatic pressure. Each scrubber 1205a, 1205b may include, as shown in FIG. 12A, an outer surface 1215 that is texturized, such as being dimpled, roughened, ridged and/or include protrusions 1220, e.g., spikes and/or bristles, in an amount, configuration and hardness to provide the desired abrasiveness or mechanical action for scrubbing. In at least some embodiments, the speed, duration, direction of the rotation can be programmed or otherwise configured to achieve a desired scrubbing intensity, which might be determined by, for example, the user, the entity operating the device (e.g., the user's employer), industry standards, or by governmental or quasi-governmental entities (CDC, WHO, OSHA, etc.).

The scrubbers may include, as does the embodiment shown in FIG. 12A, a plurality of opening, vents, nozzles, spigots and/or emitters 1225 configured to deliver to the skin sanitizing liquids, gases, UV light, ultrasound, etc., as discussed above. To deliver such liquids and gases, the openings, nozzles and spigots may be connected to the same hydraulic and pneumatic pumps that would supply such liquids and gases into the chamber as discussed above.

FIG. 12B shows the scrubbers 1205a, 1205b in use. In use, a user grasps the scrubbers 1205a, 1205b with his/her palms. The scrubbers 1205a, 1205b are then rotated, and, if so equipped/programmed, sanitizing liquids, gases, UV light, ultrasound, etc. are deliver to the user's palms. One aspect of this embodiment is that, due to the proximity of the opening, nozzles, spigots and/or emitters 1225 to the user's skin, it helps ensure delivery of the sanitizing medium where intended. The proximity also permits increased control over the intensity/force/pressure of such delivery, e.g., by controlling or setting, as applicable, the hydraulic/pneumatic pressure of the liquid or gas in the scrubber, or the intensity of the UV light or ultrasound emanating from the emitters. In some such embodiments, the delivery can be at a harsher or more intense level than provided by delivery of sanitizing medium elsewhere in the chamber.

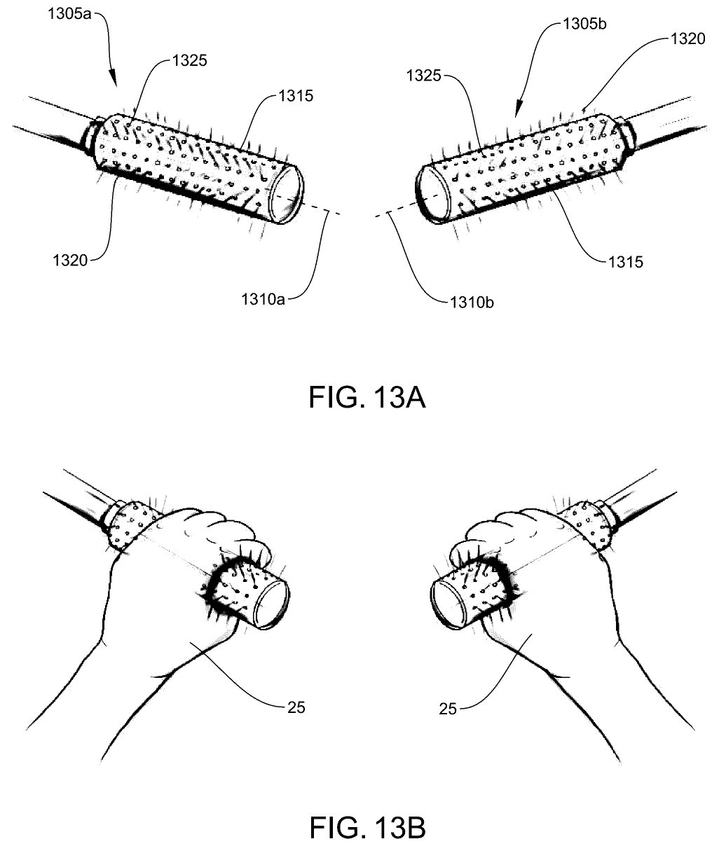

FIGS. 13A and 13B show a further embodiment having scrubbers 1305a and 1305b. Scrubbers 1305a and 1305b are similar to scrubbers 1205a and 1205b described above, and therefore like reference numerals in FIGS. 13A and 13B preceded by the numeral “13” instead of the numeral “12” are used to indicate like or similar elements. Once difference between the scrubbers 1205a and 1205b and the scrubbers 1305a and 1305b is that the latter are cylindrical or tubular in shape, as opposed to spherical. As seen in FIG. 13B, in use, the user grasps the scrubbers 1305a, 1305b with his/her palms, and the scrubbers 1305a, 1305b may be rotated and sanitizing mediums delivered to the user's skin in a similar manner as described above regarding the scrubbers 1205a, 1205b.

It should be understood that the scrubbers as shown in FIGS. 12A-13B, or variations thereof, may be implemented in any of the above-discussed systems (including the “pediatric” version), or variations thereof. At least some such systems may operate in the following exemplary manner:
(a) The device may periodically or continually check with a local or wide area network server for updates and process configuration changes as triggered by emerging contagion threats identified by hospitals, labs, and agencies such as the Center for Disease Control (CDC) and the World Health Organization (WHO), or updated industry standards or recommendations.
(b) User specific parameters are called up by the scanning of the person's credentials or identification, in order to properly adjust the unit to the specific user's height requirements, arm length and circumference in order to create the most comfortable and secure cuff seal (if present). The user's credentials would be utilized as well to set personal temperature preferences and sanitation requirements and standards that may include, for example, updated configurations based on emerging viral threat information sourced from the cloud server connected to the CDC and WHO.
(c) User would be invited to place devices or jewelry (or the like) in the accessory chamber (if present), which will be subject to similarly configured sterilizing processes or processes specifically tailored to user and application.
(d) If height adjustable, the machinery automatically adjusts to the user's height, e.g., using the wall-mounted railings or the floor mounted stand that incorporates automatic height adjustment tracks and motors. The unit's internal mechanism would alter the tunnel circumference to adjust for the user's forearm length, hand size, and forearm circumference via internal mechanical scissor type, rotary expandable, or fan type collapsible tunnel structure.
(e) If a medical/surgical application, the user places his/her arms in the machine with the hands pointed upwards. The self-sealing apertures close tightly enough around the user's forearms to create an air and water-tight seal. These cuffing seals may be, for example, inflatable pneumatic cuffs similar to those utilized in blood pressure testing equipment aided by elastic rings, which may include anti-microbial materials, e.g. anti-microbial polymers.
(f) In a non-medical configuration, the unit's form factor could be specially designed to have the hand-forearms extended out and reaching out and downwards into a cabinet without the need to be pointed upwards.
(g) A metal detecting phase that utilizes the internal metal sensors, e.g., LIDAR, magnetic, and/or Ultrasonic sensors would warn the user of any jewelry that may interfere or be damaged by the vigorous automated process via specially designed sounds, flashing lights, and/or messages on the unit screen. This would provide the user with the opportunity to withdraw from the unit and remove the jewelry, etc. and, if present, place them in the accessory chamber prior to restarting the process.
(h) The UV-A (UV-B and UV-C as alternatives) emitters turn on and optionally stay on emitting at 254 nm and 222 nm at variable durations and intervals throughout the entire cycle/process until the user has left and the unit has finalized is post-use self-cleaning routine.
(i) The process of disinfection would begin, and the UV, Ultrasound, and spigot array on the inside walls of the tunnel channels would commence their sequence, prior to or simultaneously with the array of spigots, openings, nozzles and emitters (to the extent present) on the surface of the scrubbing mechanisms.
(j) Optionally, gasses including, but not limited to ozone, nitrous oxide, and a combination of fluid antimicrobial agents such as ST37 are released by the spigots/opening/nozzles, e.g., powered by the internal pneumatic and hydraulic pumps at system-configurable pressures and varying percentages. This hydraulic and pneumatic decontamination phase would last for, for example, 2-30 seconds of variable and system configurable duration. Throughout this process both of the sealed tunnel chambers that hold the user's appendages would be filled, as well as potentially separate/accessory chambers that house apparatuses that require a pre-cleaning.
(k) The mechanisms would spin as discussed above for varying intervals while emitting UV, Ultrasound, and/or high-pressure antimicrobial gas and/or liquid mixes. The direction and interval of the mechanisms rotation and speed would be adjusted by attending staff, or automatically via the network connection to the database servers. The Ultrasound component of this sequence would emit ultrasound waves at system configurable rates and intervals that cycle through the about 20 Mhz to 100 Mhz range. The rates and intervals could be programmed to target certain pathogens and contagions, such as those identified by medical staff (through, e.g., testing/culturing) and/or based on CDC and WHO information provided by the network servers (e.g., currently-circulating variants).
(l) Optionally, Steps (h) through (k) could be repeated when a second phase of disinfection is determined to be desirable, e.g., by the hospital staff based on information about current or emerging viral and contagion threats gathered via the network as described in Step (a).
(m) A pneumatic drying stage with heated and pressurized air directed at the user's skin at high-air-speeds utilizing warm ionized air to help separate and tumble away lingering particles.
(n) Once the user's skin is determined to be suitably dry by the internal sensors, the unit suctions out of the gasses and liquids utilizing its internal pumps and replace them with regular (ambient) air.
(o) An additional sequence of UV-C and Ultrasound bursts can optionally be added if the specific medical or scientific configuration calls for it, e.g., based on the information gathered from the network connection to the CDC and WHO.
(p) An image or scan of the hands and forearms is taken using X, Y, and Z Cartesian coordinates to measure before and after bacterial count utilizing the biosensors configured to scan for and detect the presence of pathogens, contagions, or viruses.
(q) The UV-A (UV-B and UV-C as alternatives) emitters turn off.
(r) The unit releases the self-sealing apertures to allow the user to withdraw. The unit opens the accessory bin (if present), and allow the user to withdraw their possessions.
(s) The self-sealing apertures would completely close in order to conduct a self-cleaning and self-drying sequence that makes the unit aseptic and ready for the next user. This drying sequence can employ the increasing of internal pressure 3 to 5 ATM (3 to 5 times atmospheric pressure) with simultaneous irradiation of heat energy in order to evaporate and push out any lingering fluids that may have remained in the unit's various chambers post-cycle.
(t) The user-specific process and parameters may then be logged via the network onto SaaS or GME database servers so they would be available for auditing purposes in the future, tracking, and as a human resources tool. The unit could upload statistical information collected about the pathogens it encountered in the user session to the local server and/or to the global network that processes this information in conjunction with the WHO or CDC.
(u) The casing of the unit, which may or may not be made out of anti-microbial materials, such as copper or specially engineered plastics, can be subjected to the same ultrasound process in order to sanitize its exterior surface via externally-placed ultrasonic emitters. The use of UV can also be utilized (in addition or in alternative) by strategically-placed emitters and sensors, that first determine if a human is in proximity, and when such is confirmed, shower/expose the exterior of the unit with UV light.
(v) Unit goes into energy-conservation mode (e.g., sleep mode) by turning all light and sound emitters off, and (optionally) displaying a specially-designed message on the unit screen. The unit can await next user, and be called back into action (“woken up”) by detection via an external LIDAR or other proximity sensor, or by user contact with/entry into the touchscreen, utilizing a fingerprint (via a fingerprint sensor), access card, or fob swipe (e.g., RFID) to commence the new cycle.

Those of ordinary skill in the art should appreciate, though, that other embodiments may be operated in a different manner.

It should further be recognized that, via at least some embodiments, the invention can provide a portable self-contained cleaning/disinfection system. While some embodiments are installed and used in a fixed or permanent location,, e.g., a school, an industrial site, etc. other embodiments are capable of being transported to temporary sites of need, such as communities containing or at risk of infection, a construction, site, etc. In locations where grid power is unavailable or unreliable, alternate sources of electricity can be used. By way of example only, a unit may be powered by a generator, solar, wind or battery systems, or combinations thereof. In addition, antimicrobial agents and gases can be supplied by portable or semi-portable containers or tanks. When the need at a location has ended, the sanitizing unit can then be transported to another site of use.

The above describes specific examples/embodiments. The description of such embodiments should only be used to help understand the invention. That is, those skilled in the art should appreciate that the descriptions of embodiments herein are only descriptive and exemplary, and should not be interpreted as limiting the scope of the invention or the claims herein.

For example, those of ordinary skill in the art should recognize that the invention may be utilized to meet the needs of the various industries and applications, including, but not limited to: Leadership and Government—Public spaces and facilities (Court rooms, museums, parks, law enforcement and correctional facilities); Events and Conventions—Convention centers, event facilities, convert and performance venues, meeting spaces, fairgrounds, water and amusement centers, and equestrian centers; Industrial—Restaurants, Commercial Food Services and Industrial Clean Rooms; Commercial—Public Bathrooms (Gyms, Grocery Stores, Restaurants); Transportation—Airliners, train, bus, boat, ship and other Mass Transit Bathrooms; Educational—Anthropomorphic Design for Scholastic Educators and Minors; Commercial Hygiene—Hair salons, nail salons, estheticians, and other skin or hair treatment facilities; Military—Military bases, ships, offices, and other facilities; and In Home—Retail direct to consumer.

Accordingly, it should be noted that several improvements and modifications can be made to the embodiments disclosed herein by those of ordinary skill in the technical art without departing from the principles of the invention, and such that these improvements and modifications fall within the scope thereof.

What is claimed is:

1. A system comprising:
a device defining a chamber configured to receive therein a body part of a user therein and a chamber opening in communication with the chamber and configured to receive the body part therethrough for receipt of the body part into the chamber;
wherein the device includes a plurality of:
a plurality of ultraviolet light emitters configured to emit at least one sterilizing frequency of light into the chamber and irradiate the body part with said at least one sterilizing frequency of light;
a plurality of ultrasonic emitters configured to emit at least one ultrasonic sterilizing frequency into the chamber and onto the body part;
at least one vent in fluidic connection with the chamber and in fluidic connection with or fluidically connectable with at least one source of a sterilizing gas, wherein the at least one vent is configured to deliver sterilizing gas into the chamber and into contact the body part with said sterilizing gas; or
at least one nozzle in fluidic connection with the chamber and in fluidic connection with or fluidically connectable with at least one source of a sterilizing liquid, wherein the at least one nozzle is configured to deliver sterilizing liquid into the chamber and into contact the body part with said sterilizing gas.

2. A system as defined in claim 1, wherein the at least one sterilizing frequency of light comprises UV-A, UV-B and/or UV-C light.

3. A system as defined in claim 2, wherein the at least one sterilizing frequency of light comprises UV-C light.

4. A system as defined in claim 1, wherein the at least one sterilizing frequency of light comprises ultraviolet light at 222 nm and/or 254 nm.

5. A system as defined in claim 1, wherein the at least one ultrasonic sterilizing frequency comprises ultrasound between about 20 Mhz and about 100 Mhz.

6. A system as defined in claim 1, wherein the sterilizing gas comprises ozone, nitric oxide or a combination thereof.

7. A system as defined in claim 1, wherein the sterilizing liquid comprises ST37.

8. A system as defined in claim 1, wherein the device further includes a seal configured to seal the chamber and any body part within the chamber from ambient atmosphere.

9. A system as defined in claim 8, wherein the seal comprises an inflatable cuff transitionable between a deflated position permitting receipt of the body part therethrough and into the chamber and an inflated position wherein the cuff engages and seals against the body part to seal the chamber.

10. A system as defined in claim 9, wherein a surface of the cuff configured to engage the body part includes anti-microbial material.

11. A system as defined in claim 1, wherein the device includes at least one aperture in fluidic connection with the chamber and connected or connectable to a source of pressurized air and configured to deliver pressurized air into the chamber at or near the chamber opening to isolates the chamber from ambient atmosphere.

12. A system as defined claim 1, wherein the device further includes a mechanical cleaner in the chamber positioned and configured to engage a body part with the chamber and mechanically clean the body part.

13. A system as defined in claim 12, wherein the mechanical cleaner is configured to translate, vibrate and/or rotate during engagement with the body part for mechanically cleaning the body part.

14. A system as defined in claim 1, wherein the device includes a second chamber configured to receive therein an object that is not body part and a second chamber opening in communication with the second chamber and configured to receive the object therethrough for receipt of the object into the second chamber;
wherein the device includes a plurality of:
a plurality of ultraviolet light emitters configured to emit at least one sterilizing frequency of light into the second chamber and irradiate the object with said at least one sterilizing frequency of light;
a plurality of ultrasonic emitters configured to emit at least one ultrasonic sterilizing frequency into the second chamber and onto the object;
at least one vent in fluidic connection with the chamber and in fluidic connection with or fluidically connectable with at least one source of a sterilizing gas, wherein the at least one vent is configured to deliver sterilizing gas into the second chamber and into contact the object with said sterilizing gas; or
at least one nozzle in fluidic connection with the chamber and in fluidic connection with or fluidically connectable with at least one source of a sterilizing liquid, wherein the at least one nozzle is configured to deliver sterilizing liquid into the second chamber and into contact the body part with said sterilizing gas.

15. A system as defined in claim 1, wherein the device further includes a biosensor configured to detect a pathogen on the body part.

16. A system as defined in claim 15, wherein the system is configured to deliver into the chamber and onto the body part a combination of said at least one sterilizing frequency of light, at least one ultrasonic sterilizing frequency, sterilizing gas or sterilizing liquid configured to neutralize a pathogen detected by the biosensor.

17. A system as defined in claim 15, wherein the system is configured to transmit information regarding a pathogen detected by the biosensor to a device located on the Internet or the Cloud.

18. A method comprising:
receiving within a chamber a body part of a user;
delivering into the chamber and onto the body part a plurality of:
at least one sterilizing frequency of light;
at least one ultrasonic sterilizing frequency;
at least one sterilizing gas; or
at least one sterilizing liquid.

19. A method as defined in claim 18, wherein the at least one sterilizing frequency of light comprises UV-A, UV-B and/or UV-C light.

20. A method as defined in claim 19, wherein the at least one sterilizing frequency of light comprises UV-C light.

21. A method as defined in claim 18, wherein the at least one sterilizing frequency of light comprises ultraviolet light at 222 nm and/or 254 nm.

22. A method as defined in claim 18, wherein the at least one ultrasonic sterilizing frequency comprises ultrasound between about 20 Mhz and about 100 Mhz.

23. A method as defined in claim 18, wherein the sterilizing gas comprises ozone, nitric oxide or a combination thereof.

24. A method as defined in claim 18, wherein the sterilizing liquid comprises ST37.

25. A method as defined in claim 18, further including sealing the chamber and any body part within the chamber from ambient atmosphere.

26. A method as defined in claim 25, wherein the sealing step includes inflating an inflatable cuff and engaging and sealing the cuff against the body part and thereby seal the chamber from ambient atmosphere.

27. A method as defined in claim 26, wherein a surface of the cuff engaging the body part includes anti-microbial material.

28. A method as defined in claim 25, wherein the sealing step includes delivering pressurized air into the chamber at or near the chamber opening and isolating the chamber from ambient atmosphere with the pressurized air.

29. A method as defined in claim 18, further including engaging a body part within the chamber with a cleaner and mechanically cleaning the body part.

30. A method as defined in claim 18, wherein the mechanical cleaning includes translating, vibrating and/or rotating the cleaner during said engaging of the body part with said cleaner.

31. A method as defined in claim 18, further including
receiving within a second chamber an object and
delivering into the second chamber and onto the object a plurality of:
at least one sterilizing frequency of light;
at least one ultrasonic sterilizing frequency;
at least one sterilizing gas; or
at least one sterilizing liquid.

32. A method as defined in claim 18, further including scanning for a pathogen on the body part with a biosensor.

33. A method as defined in claim 32, including delivering into the chamber and onto the body part a combination of said at least one sterilizing frequency of light, at least one ultrasonic sterilizing frequency, sterilizing gas or sterilizing liquid configured to neutralize a pathogen detected by the biosensor.

34. A method as defined in claim 32, further including transmitting information regarding a pathogen detected by the biosensor to a device located on the Internet or the Cloud.
